(19) United States
(12) Patent Application Publication
(10) Pub. No.: US 2026/0207147 A1
(43) Pub. Date: Jul. 23, 2026

(54) Mobile Medical Imaging System Including A Latching System

(71) Applicant: Mobius Imaging, LLC, Shirley, MA (US)

(72) Inventor: Russell Stanton, Lunenberg, MA (US)

(73) Assignee: Mobius Imaging, LLC, Shirley, MA (US)

(21) Appl. No.: 19/138,286

(22) PCT Filed: Dec. 12, 2023
(86) PCT No.: PCT/US2023/083526

§ 371 Date: Jun. 12, 2025

Related U.S. Application Data
(60) Provisional application No. 63/431,889, filed on Dec. 12, 2022.

(51) Int. Cl. A61B 6/10; A61B 6/00; A61B 6/02; A61B 6/03; A61B 6/40; A61B 34/30

(52) U.S. Cl. CPC A61B 6/105; A61B 6/027; A61B 6/035; A61B 6/4078; A61B 6/4447; A61B 6/4476; A61B 34/30

(57) ABSTRACT

A mobile medical imaging system includes a base defining a track, an imaging gantry, and a gantry mount supporting the imaging gantry for movement along the track between a plurality of track poses. The plurality of track poses includes a park pose defined with the gantry mount arranged adjacent to a first track end. The mobile medical imaging system further includes a catch operatively attached to the gantry mount and a latching system including a pedal operatively attached to the base and supporting a latch. The pedal is configured for movement between an engaged position that places the latch in a lock position with the latch engaging the catch to retain the imaging gantry in the park pose, and a disengaged position that places the latch in a released position with the latch spaced from the catch to permit translation of the gantry mount away from the park pose.

### CROSS-REFERENCE TO RELATED APPLICATION

The subject patent application claims priority to and all the benefits of U.S. Provisional Patent Application No. 63/431,889 filed on Dec. 12, 2022, the disclosure of which is hereby incorporated by reference in its entirety.

### BACKGROUND

Imaging gantries that are supported along a base for translation must be back-drivable in case the imaging system loses power in order to allow the imaging gantry to be translated away from a patient to remove the patient from the imaging system. However, the back-drivable nature of the structure supporting the imaging gantry may cause the imaging gantry to inadvertently back-drive when the intention is for the imaging gantry to remain stationary. Accordingly, there remains a need in the art for addressing one or more of these deficiencies.

### SUMMARY

One general aspect of the present disclosure includes a mobile medical imaging system. The mobile medical imaging system includes a base defining a track extending between a first track end and a second track end, an imaging gantry having at least one imaging component and defining an imaging bore, and a gantry mount supporting the imaging gantry for movement along the track between a plurality of track poses. The plurality of track poses includes a park pose defined with the gantry mount arranged adjacent to the first track end. The mobile medical imaging system also includes a translation mechanism interposed between the base and the imaging gantry to drive the gantry mount between the plurality of track poses in an imaging mode to acquire image data of a patient within the imaging bore. The mobile medical imaging system further includes a catch operatively attached to the gantry mount for concurrent movement between the plurality of track poses. The mobile medical imaging system also further includes a latching system including a pedal operatively attached to the base and supporting a latch. The pedal is configured for movement between: an engaged position that places the latch in a lock position with the latch engaging the catch to retain the imaging gantry in the park pose, and a disengaged position that places the latch in a released position with the latch spaced from the catch to permit translation of the gantry mount away from the park pose in response to one of: powered operation of the translation mechanism, and user-applied force applied to back-drive the translation mechanism.

### BRIEF DESCRIPTION OF THE DRAWINGS

Other advantages of the present invention will be readily appreciated, as the same becomes better understood by reference to the following detailed description when considered in connection with the accompanying drawings.

FIG. 1A is a top perspective view of a mobile medical imaging system according to the present disclosure and including a base, a gantry mount in a park pose, and an imaging gantry.

FIG. 1B is a top perspective view of a mobile medical imaging system of FIG. 1A with the gantry mount and an imaging gantry translated along the base in an imaging mode.

FIGS. 2A-2C show various examples of the imaging gantry including at least one imaging component.

FIG. 3 is a schematic representation of the mobile medical imaging system performing a helical scanning procedure.

FIG. 4 is a top perspective view of a schematic representation of the mobile medical imaging system including a gimbal with the imaging gantry tilted relative to the gimbal.

FIGS. 5A and 5B are side and top schematic views, respectively, of the mobile medical imaging system in an imaging mode and the gantry mount in a park pose.

FIGS. 6A and 6B are side and top schematic views, respectively, of the imaging system of FIGS. 5A and 5B in a transport mode and the gantry mount in a transport pose.

FIG. 7 is a representation of an operating room including the mobile medical imaging system and a robotic surgical system.

FIG. 8A is a partial bottom perspective view of the mobile medical imaging system with the gantry mount in the park pose and including a catch operatively attached to the imaging gantry and a latching system including a pedal in a disengaged position supporting a latch in a released position where the latch is spaced from the catch.

FIG. 8B is a partial bottom perspective view of the mobile medical imaging system with the gantry mount in the park pose and the pedal in an engaged position supporting the latch in a lock position where the latch engages the catch to retain the gantry mount in the park pose.

FIG. 9A is a partial cross-section representation of the mobile medical imaging system taken through the latch to reveal the pedal is in the disengaged position supporting the latch in the released position relative to a bottom portion of the imaging gantry.

FIG. 9B is a partial cross-section representation of the mobile medical imaging system taken through the latch to reveal the latch relative to a bottom portion of the imaging gantry as the pedal moves toward the engaged position and a first profile of the catch cooperates with a second profile of the latch to urge the gantry mount to the park pose.

FIG. 9C is a partial cross-section representation of the mobile medical imaging system taken through the latch to reveal the pedal is in the disengaged position supporting the latch in the released position relative to a bottom portion of the imaging gantry.

FIG. 10A is an enlarged view of FIG. 9A.

FIG. 10B is an enlarged view of FIG. 9B.

FIG. 10C is an enlarged view of FIG. 9C.

FIG. 11A shows a cross-sectional representation of the latching system with the pedal in the engaged position taken through a biasing member of the latching system to reveal the biasing member and a link for retaining the pedal in the disengage position.

FIG. 11B shows a cross-sectional representation of the latching system as the pedal moves toward the disengaged position and the link traveling along a first channel in response to a first user engagement with the pedal.

FIG. 11C shows a cross-sectional representation of the latching system as the pedal reaches the disengaged position and the link abuts a link landing to retain the pedal in the disengaged position.

FIG. 11D shows a cross-sectional representation of the latching system with the link entering a second channel in response to a second user engagement with the pedal.

FIG. 11E shows a cross-sectional representation of the latching system as the pedal moves toward the engaged position and the link traveling along the first channel in response to the second user engagement with the pedal.

FIG. 11F shows a cross-sectional representation of the latching system with the pedal returning to the engaged position and the link returning to the first channel.

FIG. 12A is a partial schematic side view of the mobile medical imaging system including a gantry motor, a translation motor, a gimbal motor, and the imaging gantry in communication with a system controller, and the gantry mount and the imaging gantry arranged between a first track end and a second track end and the pedal of the latching system in the engaged position.

FIG. 12B is a partial schematic side view of the mobile medical imaging system with the gantry mount and the imaging gantry translating toward the first track end and a bottom portion of the gantry mount deflecting the pedal toward the disengaged position.

FIG. 12C is a partial schematic side view of the mobile medical imaging system with the gantry mount and the imaging gantry translating in the park pose and the pedal of the latching system in the engage position to retain the gantry mount in the park pose.

### DETAILED DESCRIPTION

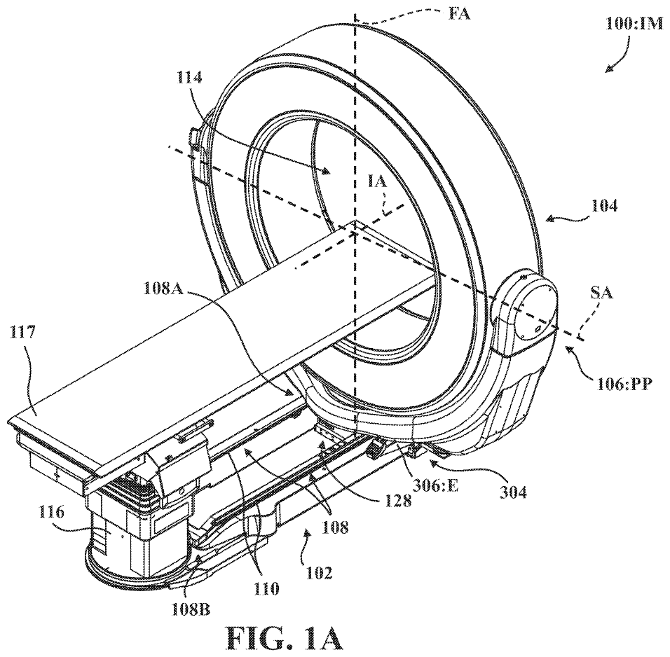

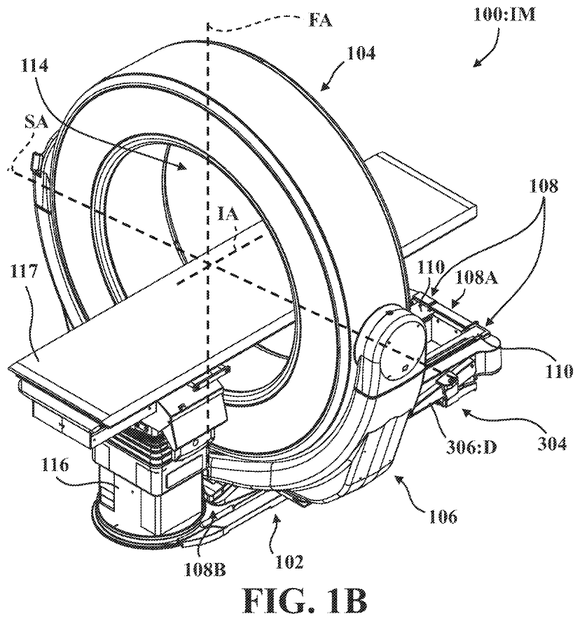

FIGS. 1A-1B generally show a mobile medical imaging system 100 according to the present disclosure. The mobile medical imaging system 100 generally includes a base 102, an imaging gantry 104, and a gantry mount 106. The base 102 defines a track 108 extending between a first track end 108A and a second track end 108B. In one example, the track 108 may be defined by a pair of rails 110 that are spaced apart from and parallel to each other. As will be described in further detail below, the gantry mount 106 supports the imaging gantry 104 for movement along the track 108.

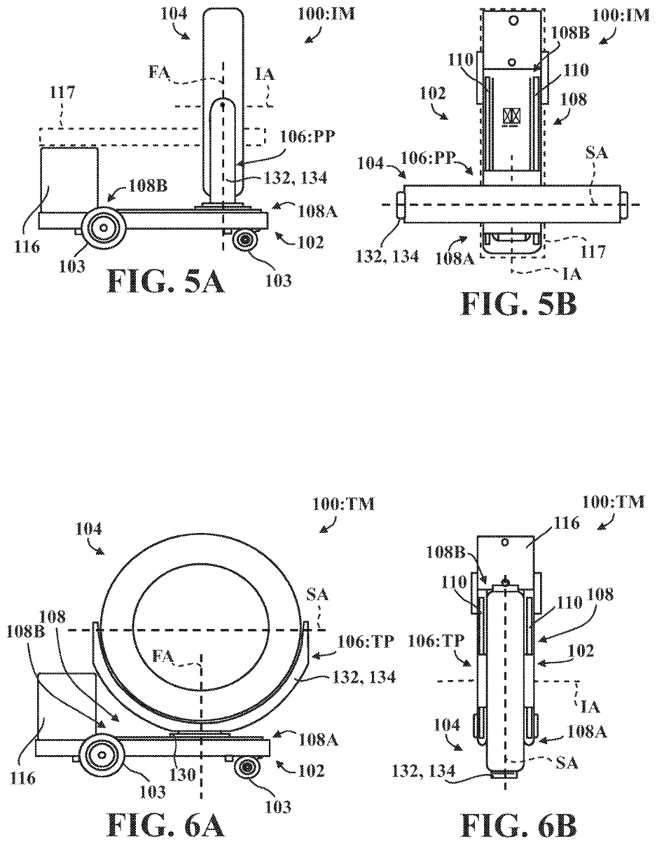

The base 102 is generally mobile relative to floor surfaces such that the mobile medical imaging system 100 is mobile relative to floor surfaces. For example, the base 102 may include a generally rectangular housing having a length and width preferably designed to allow the mobile medical imaging system 100 to fit through most standard-sized doorways (i.e., generally 24-36 inches wide), and to be easily transported through corridors and elevators generally found in hospitals and other healthcare environments. To facilitate movement of the mobile medical imaging system 100 over floor surfaces, the base 102 may include one or more wheels 103 (best shown in FIGS. 5A and 6A). The base 102 may also include a base lift (not shown) interposed between the base housing and the one or more wheels 103 and operable to lift and lower the base 102 relative to floor surfaces. In one example, the one or more wheels 103 may be casters that extend relative to the base 102 to lift the base 102 off the ground in a transport mode TM to allow the base 102 to be moved relative to floor surfaces, and retract relative to the base 102 to lower the base onto floor surfaces in an imaging mode IM. One exemplary configuration of a base 102 that is mobile relative to floor surfaces and includes a base lift is disclosed in U.S. Patent Application Publication No. U.S. 2022/0061779 entitled “Caster System For Mobile Apparatus,” which is incorporated by reference herein in its entirety. It is also contemplated that the mobile medical imaging system 100 may also include a transport motor that is geared with the one or more wheels 103 to propel the mobile medical imaging system 100 across floor surfaces. The mobile medical imaging system 100 may also include a steering mechanism for guiding the direction of the mobile medical imaging system 100 over floor surfaces. Other configurations enabling the base 102 to be mobile relative to floor surfaces FS are contemplated.

The imaging gantry 104 generally includes at least one imaging component 112 and defines an imaging bore 114 defining an imaging axis IA. The mobile medical imaging system 100 is configured to collect imaging data ID, such as, for example x-ray computed tomography (CT) or magnetic resonance imaging (MRI) data, from an object located within the imaging bore 114 of the imaging gantry 104, in any manner known in the medical imaging field. An exemplary imaging gantry 104 that may be used in various versions is the AIRO® intra-operative CT system manufactured by Mobius Imaging, LLC. Examples of x-ray CT imaging devices that may be used according to various versions of the present disclosure are described in U.S. Pat. No. 10,151,810, entitled “Pivoting Multi-directional X-ray Imaging System with a Pair of Diametrically Opposite Vertical Support Columns Tandemly Movable Along a Stationary Base Support;” U.S. Pat. No. 9,962,132, entitled “Multi-directional X-ray Imaging System with Single Support Column;” U.S. Pat. No. 9,801,592, entitled “Caster System for Mobile Apparatus;” U.S. Pat. No. 9,111,379, entitled “Method and System for X-ray CT Imaging;” U.S. Pat. No. 8,118,488, entitled “Mobile Medical Imaging System and Methods;” and U.S. Patent Application Publication No. 2014/0275953, entitled “Mobile X-ray Imaging System,” the disclosures of each of which are hereby incorporated by reference in their entirety.

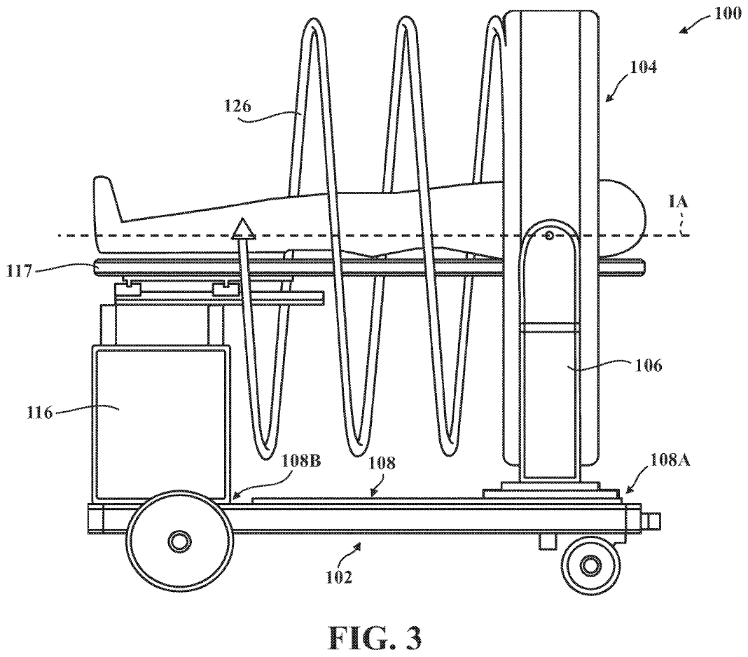

As shown in FIGS. 1A-1B, the mobile medical imaging system 100 may include a pedestal 116. The pedestal 116 may extend generally vertically upwards from the base 102 and be mounted to the base 102 adjacent to the second track end 108B to support a patient P above the base 102 (best shown in FIG. 3). For example, the pedestal 116 may be adapted to support a patient support 117 that can be attached to the pedestal 116. In one example, the patient support 117 is mounted to the pedestal 116 in a cantilevered manner and extends out into the imaging bore 114 of the imaging gantry 104 to support a patient P or other object being imaged. It will be understood that virtually any type of patient support 117 can be used in the present imaging system. For example, the present imaging system can utilize medical tables, and related accessories, of the type described in the JUPITER system brochure (11/2008) from TRUMPF Medezin Systeme GmbH & Co. KG of Puchheim, Germany, the entire contents of which are incorporated herein by reference. Furthermore, although the present examples illustrate patient supports 117 that can be used for medical imaging of human patients, it will be understood that the present invention encompasses any suitable tabletop support structure, including those designed for or suitable to support non-human subjects and non-living objects and materials. A plurality of different patient supports 117 can be attached and detached from the pedestal, where the tabletop supports are each customized for a particular application. Examples of different configurations of the pedestal 116 and patient support 117 are described in U.S. Patent Application Publication No. 2021/006,8775 entitled “Medical Imaging System and Methods,” the disclosure of which is hereby incorporated by reference in its entirety.

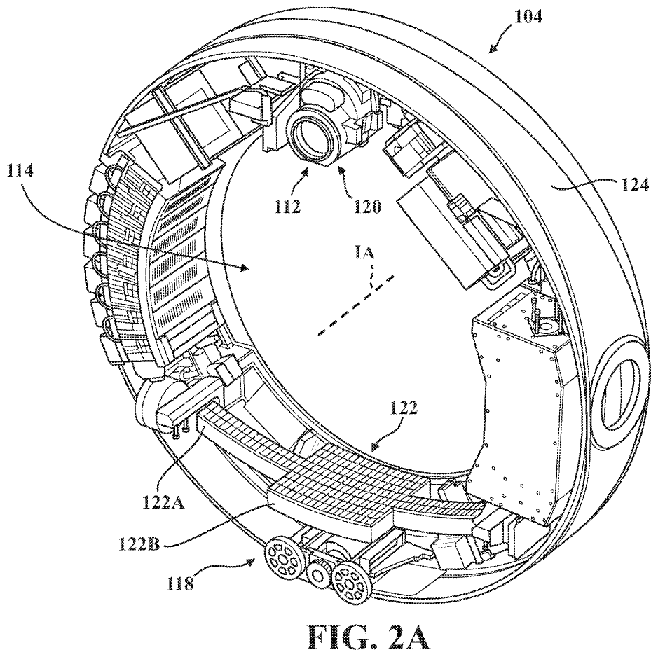

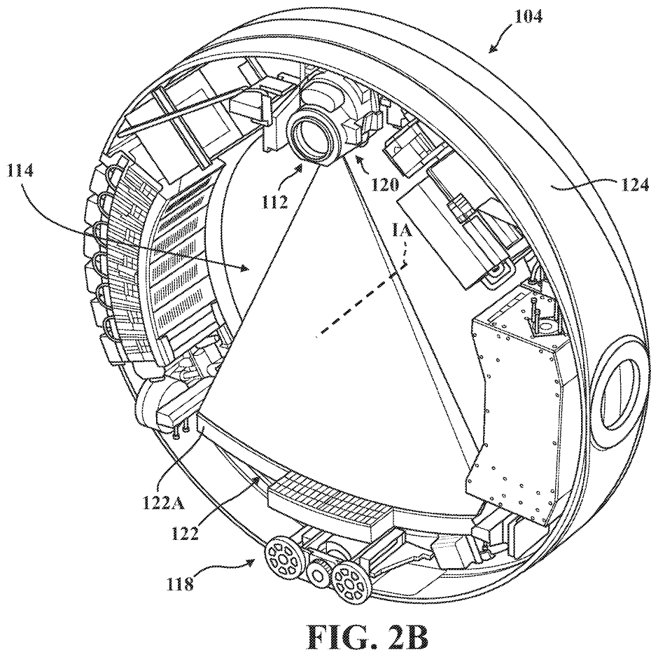

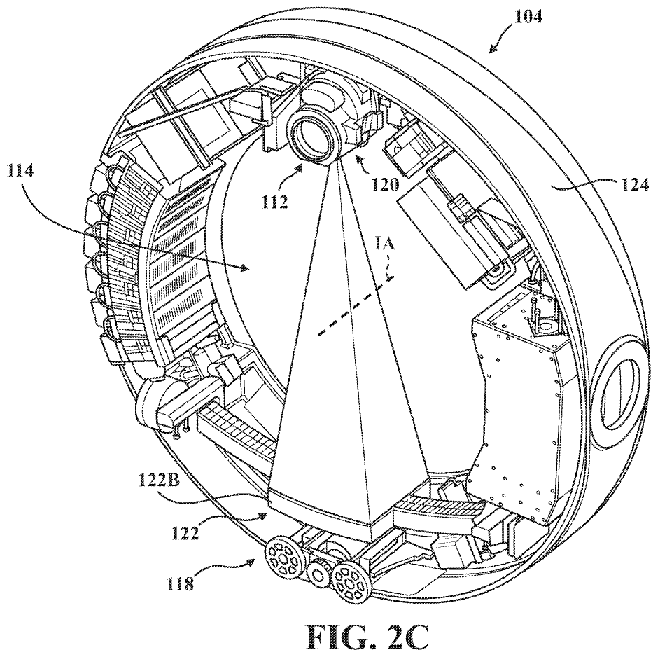

FIGS. 2A-2C show one example of the imaging gantry 104 including the at least one imaging component 112. In this example, the at least one imaging component 112 of the imaging gantry 104 includes a rotor 118 supporting an x-ray source 120 and a detector 122. The rotor 118 may be disposed within a gantry housing 124 defined by the imaging gantry 104 for rotation around the imaging bore 114. In some examples, the x-ray source 120 may be a fan-beam x-ray source (shown in FIG. 2B) and/or a cone-beam x-ray source (shown in FIG. 2C). The detector 122 may comprise an array of detectors 122. The array of detectors 122 may define an elongated first portion 122A for performing fan-beam CT imaging (e.g., axial and/or helical scans), a panel-shaped second portion 122B for performing 2D fluoroscopic imaging and/or 3D cone beam CT imaging, or a combination thereof, as shown in FIGS. 2A and 2B. FIG. 3 illustrates an example of the mobile medical imaging system 100 performing a helical scan. Here, the rotor 118 supporting the x-ray source 120 and the array of detectors 122 rotate around the imaging bore 114 of the gantry 104 to obtain imaging data, while the imaging gantry 104 and gantry mount 106 simultaneously translate along the base 102 from the first track end 108A to the second track end 108B (described in further detail below). The arrow 126 indicates the path of the at least one imaging component 112 around the imaging bore 114 in a helical scan.

Referring back to FIGS. 1A and 1B, as described above, the gantry mount 106 supports the imaging gantry 104 for movement along the track 108. The gantry mount 106 may be configured for movement between a plurality of track poses. The plurality of track poses may include a park pose PP, shown in FIG. 1A. In the park pose PP, the gantry mount is arranged adjacent to the first track end 108A. The mobile medical imaging system 100 also includes a translation mechanism 128 interposed between the base 102 and the imaging gantry 104 to drive the gantry mount 106 between the plurality of track poses in the imaging mode IM to acquire image data ID of a patient within the imaging bore 114, as shown in FIG. 3.

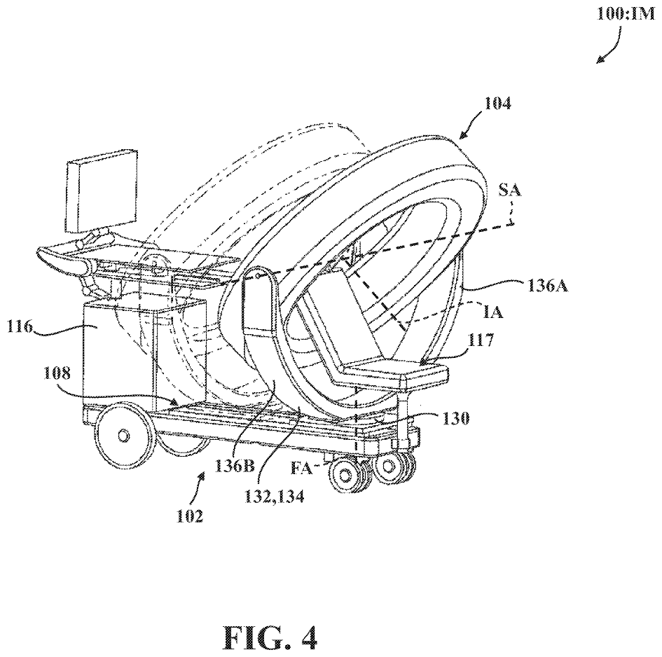

In some configurations, as best shown in FIGS. 4-6B, the gantry mount 106 includes a gantry mount base 130 that is operatively attached to the base 102, and a gantry mount member 132 operatively attached to the gantry mount base 130 for rotation relative to the gantry mount base 130 about a first axis FA. In these configurations, the gantry mount member 132 supports the imaging gantry 104 such that the gantry mount member 132 and the imaging gantry 104 are configured to rotate together about the first axis FA relative to the base 102. In some examples, the gantry mount member 132 includes a gimbal 134 having a pair of arms 136A, 136B (best shown in FIG. 4). Each of the arms 136A, 136B is coupled to an opposite side of the imaging gantry 104 to support the imaging gantry 104 above the base 102 and the gimbal 134. Additionally, referring to FIG. 4, in some configurations, the imaging gantry 104 may be configured to tilt about a second axis SA relative to the gimbal 134. Advantageously, allowing the imaging gantry 104 to tilt about the second axis SA may provide a number of different imaging configurations, such as shown in FIG. 4,

FIGS. 5A and 5B are side and top views, respectively, of the mobile medical imaging system 100 in the imaging mode IM and the gantry mount 106 in the park pose PP. In the park pose PP, the imaging axis IA is parallel to the track 108 and the gantry mount 106 is arranged adjacent to the first track end 108A. FIGS. 6A and 6B, show the mobile medical imaging system 100 in a transport mode TM and the gantry mount 106 in a transport pose TP. In the transport mode TM, the pedestal 116 and/or the patient support 117 may be removed from the mobile medical imaging system 100. In the transport pose TP, the gantry mount 106 is arranged between the first track end 108A and the second track end 108B and the gantry mount member 132 and the imaging gantry 104 are rotated about the first axis FA such that the imaging axis IA is transverse to the track 108. The profile of the mobile medical imaging system 100 is thus dramatically reduced in comparison to the imaging mode, such that the mobile medical imaging system 100 in the transport mode TM is typically only as wide as the width of the base 102 and/or the pedestal 116. This advantageously allows the system 100 to be more easily transported through narrow doors and hallways. The imaging system 100 can easily switch between the imaging mode IM and the transport mode TM, and vice versa, by rotating the imaging gantry 104 and gantry mounting member 132 with respect to the base 102.

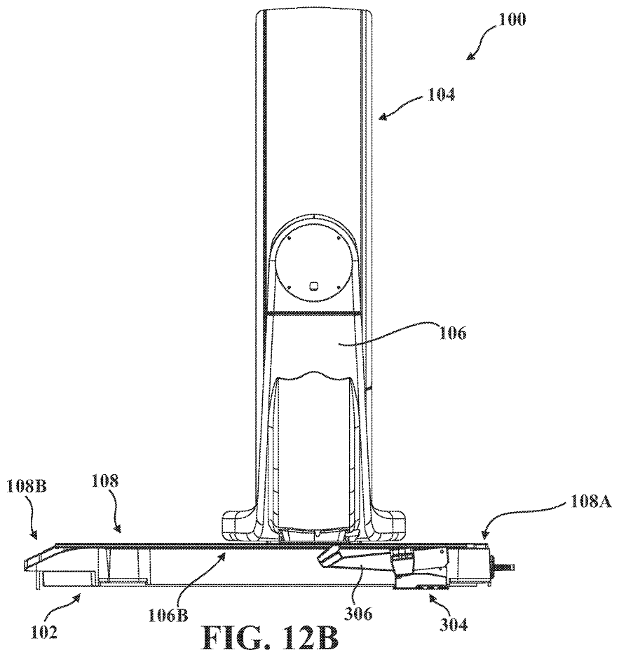

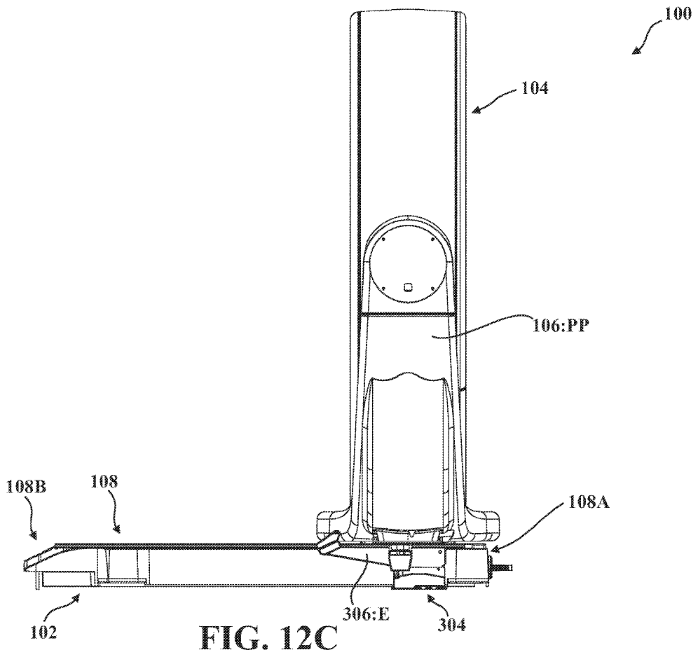

The mobile medical imaging system 100 can include one or more motors, as are known in the art, to control and effect the above-described motions. For example, as illustrated schematically in FIG. 12A, the mobile medical imaging system 100 may include a translation motor 138 operatively attached to the translation mechanism 128 to drive the gantry mount 106 between the plurality of track poses, and a gantry motor 140 operatively attached to the gantry mount base 130 and the gantry mount member 132 for rotating the gantry mount member 132 relative to the base 102 about the first axis FA, and a gimbal motor 142 interposed between the gimbal 134 and the imaging gantry 104 for rotating the imaging gantry 104 relative to the gimbal 134 about the second axis SA. All of these respective motions can be controlled by a central computerized system controller 144. The system controller 144 may be included on the mobile medical imaging system 100, such as housed inside the pedestal 116. In other examples, the system controller 144 may be located off the mobile medical imaging system 100, such as in a mobile cart, and may comprise a general purpose computer programmed to provide the desired control functions and user interface, and is in electrical communication with the mobile medical imaging system 100, such as via a cable or wireless link. The control system 144 can also control the operation of the at least one imaging component of the imaging gantry 104.

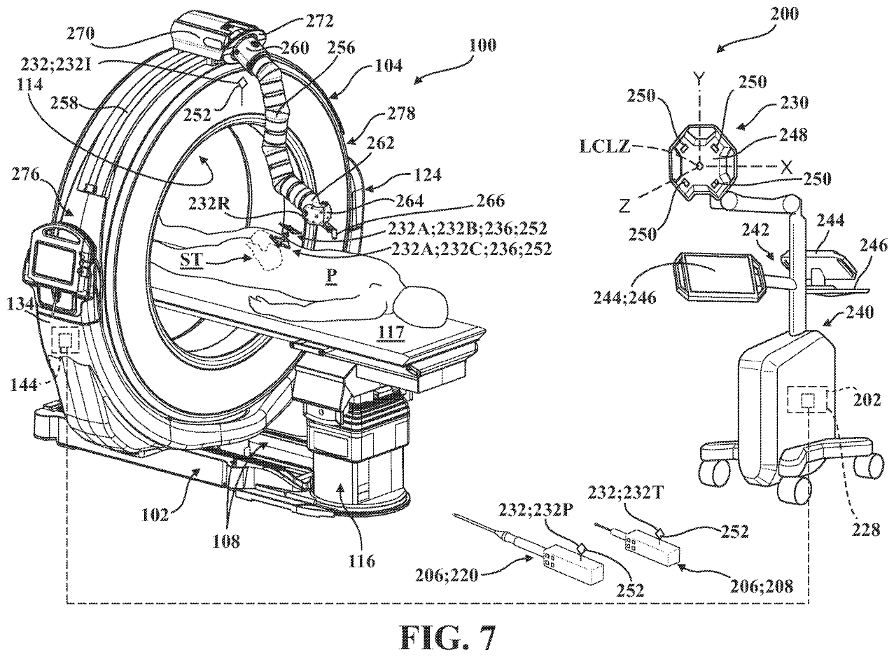

Referring to FIG. 7, the mobile medical imaging system 100 may further include a robotic system 200 for treating a patient P. The illustrated robotic system 200 generally includes a navigation system 202 one or more types of tools 206. As will be appreciated from the subsequent description below, the robotic system 200 is configured to, among other things, allow the surgeon to visualize, approach, and treat or otherwise manipulate anatomy of a patient P at a target site ST with a high level of control. To this end, imaging data ID of the target site ST may be acquired via the mobile medical imaging system 100, and can be used to assist the surgeon in visualizing the patient's P anatomy at or otherwise adjacent to the target site ST. Here, the imaging data ID may also be utilized by the navigation system 202 to, among other things, facilitate navigation of tools 206 relative to the target site ST. Each of the components of the robotic system 200 introduced above will be described in greater detail below.

In FIG. 7, an operating room is shown with a patient P undergoing an exemplary surgical procedure performed using the robotic system 200. In this illustrative example, a minimally-invasive spinal surgical procedure, such as a posterior interbody spinal fusion, is being performed. It will be appreciated that this example is illustrative, and that other types of surgical procedures are contemplated. During the surgical procedure, one or more hand-held tools 206, such as a rotary tool 208 and/or a pointer tool 210, may be used by the surgeon. The tool 206 is for engaging the target site ST. As noted above and as is described in greater detail below, the navigation system 202 may be configured to track states of one or more of the tools 206 relative to the target site ST. In this exemplary surgical procedure, the rotary tool 208 may be employed as a cutting or drilling tool to remove tissue, form pilot holes (e.g., in the ilium, in vertebrae, and the like), or otherwise approach the target site ST. The rotary tool 208 may also be used to drive or otherwise install implantable components (e.g., pedicle screws, anchors, and the like).

For illustrative purposes, generically-depicted tools 206 configured for hand-held use are shown in FIG. 7. However, as will be appreciated from the subsequent description below, aspects of the robotic system 200 may be used with any suitable type of tool 206 without departing from the scope of the present disclosure. Furthermore, in addition to hand-held tools 206 of various types and configurations, aspects of the robotic system 200 may also be employed in connection with robotically-controlled tools 206 (not shown). Certain types of robotically-controlled tools 206 are disclosed in U.S. Pat. No. 9,119,655, entitled “Surgical Robotic arm Capable of Controlling a Surgical Instrument in Multiple Modes;” U.S. Pat. No. 10,456,207, entitled “Systems and Tools for use with Surgical Robotic Manipulators;” U.S. Pat. No. 11,160,620, entitled “End Effectors And Methods For Driving Tools Guided By Surgical Robotic Systems;” U.S. Pat. No. 10,959,783, entitled “Integrated Medical Imaging and Surgical Robotic System;” and U.S. Patent Application Publication No. 2020/0078097, entitled “Methods and Systems for Robot-Assisted Surgery,” the disclosures of each of which are hereby incorporated by reference in their entirety.

As noted above, the mobile medical imaging system 100 may be used to obtain imaging data ID of the patient, which may be a human or animal patient. In the representative version illustrated in FIG. 7, the mobile medical imaging system 100 is realized as an x-ray computed tomography (CT) imaging device configured to obtain raw x-ray imaging data ID of the patient P, as described above. The imaging data ID may be processed using the system controller 144, or another suitable controller, in order to construct three-dimensional imaging data ID, two-dimensional imaging data ID, and the like, which may be transmitted to or otherwise utilized by the navigation system 202 or other components of the robotic system 200.

In some versions, imaging data ID may be obtained preoperatively (e.g., prior to performing a surgical procedure) or intraoperatively (e.g., during a surgical procedure) by positioning the patient P within the imaging bore 114 of the mobile medical imaging system 100. In order to obtain imaging data ID, a portion of the mobile medical imaging system 100 may be moved relative to the patient support 117 (described above) on which the patient P is disposed.

The robotic system 200 employs the navigation system 202 to, among other things, track movement of various objects, such as the tools 206 and parts of the patient's P anatomy (e.g., tissue at the surgical site ST), as well as portions of the mobile medical imaging system 100 in some versions. To this end, the navigation system 202 comprises a navigation controller 228 coupled to a localizer 230 that is configured to sense the position and/or orientation of trackers 232 within a localizer coordinate system LCLZ. In other words, the navigation system 202 includes the localizer 230 to track states of trackers 232 within a field of view. As is described in greater detail below, the trackers 232 (also referred to herein as “navigable trackers”) are fixed, secured, or otherwise attached to specific objects, and are configured to be monitored by the localizer 230.

The navigation controller 228 is disposed in communication with the localizer 230 and gathers position and/or orientation data for each tracker 232 sensed by the localizer 230 in the localizer coordinate system LCLZ. The navigation controller 228 may be disposed in communication with the system controller 144 e.g., to receive imaging data ID) and/or in communication with other components of the robotic system 200 (e.g., robotic arm controllers, tool controllers, and the like; not shown). However, other configurations are contemplated. The controllers 144, 228 may be realized as computers, processors, control units, and the like, and may be discrete components, may be integrated, and/or may otherwise share hardware.

It will be appreciated that the localizer 230 can sense the position and/or orientation of multiple trackers 232 to track correspondingly multiple objects within the localizer coordinate system LCLZ. By way of example, and as is depicted in FIG. 7, trackers 232 may comprise a tool tracker 232T, a pointer tracker 232P, an imaging system tracker 232I, one or more patient trackers 232A (e.g., a first patient tracker 232A, a second patient tracker 232B, and the like), a robot tracker 232R, as well as additional patient trackers, trackers for additional medical and/or surgical tools, and the like. The patient tracker 232A is adapted for attachment relative to the target site ST. One example of the robot tracker 232R is described in U.S. Provisional Patent Application 63/348,115 entitled “Robotic Surgical System with End Effector Marker Diffusers” which is incorporated by reference herein in its entirety.

The position of the patient trackers 232A, 232B relative to the anatomy of the patient P to which they are attached can be determined by known registration techniques, such as point-based registration in which the pointer tool 210 (to which the pointer tracker 232P is fixed) is used to touch off on bony landmarks on bone, or to touch off on several points across the bone for surface-based registration. Conventional registration techniques can be employed to correlate the pose of the patient trackers 232A, 232B to the patient's anatomy. Other types of registration are also possible.

Position and/or orientation data may be gathered, determined, or otherwise handled by the navigation controller 228 using conventional registration/navigation techniques to determine coordinates of trackers 232 within the localizer coordinate system LCLZ. These coordinates may be utilized by various components of the robotic system 200 (e.g., to facilitate control of the tools 206, to facilitate navigation based on imaging data ID, and the like).

In the representative version illustrated in FIG. 7, the navigation controller 228 and the localizer 230 are supported on a mobile cart 240 which is movable relative to the base 102 of the mobile medical imaging system 100. The mobile cart 240 also supports a user interface, generally indicated at 242, to facilitate operation of the navigation system 202 by displaying information to, and/or by receiving information from, the surgeon or another user. The user interface 242 may be disposed in communication with other components of the robotic system 200 (e.g., with the mobile medical imaging system 100), and may comprise one or more output devices 244 (e.g., monitors, indicators, display screens, and the like) to present information to the surgeon (e.g., images, video, data, a graphics, navigable menus, and the like), and one or more input devices 246 (e.g., buttons, touch screens, keyboards, mice, gesture or voice-based input devices, and the like).

In some versions, the robotic system 200 is capable of displaying a virtual representation of the relative positions and orientations of tracked objects to the surgeon or other users of the robotic system 200, such as with images and/or graphical representations of the anatomy of the patient P and the tool 206 presented on one or more output devices 244 (e.g., a display screen). The navigation controller 228 may also utilize the user interface 242 to display instructions or request information from the surgeon or other users of the robotic system 200. Other configurations are contemplated. One type of mobile cart 240 and user interface 242 of this type of navigation system 202 is described in U.S. Pat. No. 7,725,162, entitled “Surgery System,” the disclosure of which is hereby incorporated by reference in its entirety.

Because the mobile cart 240 and the imaging gantry 104 of the mobile medical imaging system 100 can be positioned relative to each other and also relative to the patient P in the representative version illustrated in FIG. 7, the navigation system 202 can transform the coordinates of each tracker 232 from the localizer coordinate system LCLZ into other coordinate systems (e.g., defined by different trackers 232, localizers 230, and the like), or vice versa, so that navigation relative to the target site ST (or control of tools 206) can be based at least partially on the relative positions and orientations of multiple trackers 232 within a common coordinate system (e.g., the localizer coordinate system LCLZ). Coordinates can be transformed using a number of different conventional coordinate system transformation techniques. It will be appreciated that the localizer 230 or other components of the navigation system 202 could be arranged, supported, or otherwise configured in other ways without departing from the scope of the present disclosure. By way of non-limiting example, the localizer 230 could be coupled to the mobile medical imaging system 100 in some versions (e.g., to the imaging gantry 104). Other configurations are contemplated.

In the illustrated version, the localizer 230 is an optical localizer and includes a camera unit 248 with one or more optical position sensors 250. The navigation system 202 employs the optical position sensors 250 of the camera unit 248 to sense the position and/or orientation of the trackers 232 within the localizer coordinate system LCLZ. To this end, the trackers 232 each employ one or more markers 252 (also referred to as “fiducials” in some versions) that are supported on an array in a predetermined arrangement. However, as will be appreciated from the subsequent description below, trackers 232 may have different configurations, such as with different quantities of markers 252 that can be secured to or otherwise formed in other structures besides arrays (e.g., various types of housings, frames, surfaces, and the like). Other configurations are contemplated.

In some versions, certain trackers 232 (e.g., the patient tracker 232A) may employ “passive” markers 252 (e.g., reflective markers such as spheres, cones, and the like) which reflect emitted light that is sensed by the optical position sensors 250 of the camera unit 248. In some versions, trackers 232 employ “active” markers 252 (e.g., light emitting diodes “LEDs”), which emit light that is sensed by the optical position sensors 250 of the camera unit 248. Examples of navigation systems 202 of these types are described in U.S. Pat. No. 9,008,757, entitled “Navigation System Including Optical and Non-Optical Sensors,” the disclosure of which is hereby incorporated by reference in its entirety.

Although one version of the mobile cart 240 and localizer 230 of the navigation system 202 is illustrated in FIG. 7, it will be appreciated that the navigation system 202 may have any other suitable configuration for monitoring trackers 232 which may be of various types and configurations and could employ various types of markers 252. Thus, for the purposes of clarity and consistency, the term “marker 252” is used herein to refer to a portion of a tracker 232 (e.g., a passive or active marker 252 mounted to an array or otherwise coupled to a tracked object) that can be monitored by a localizer 230 to track (e.g., states, motion, position, orientation, and the like) of the object to which the tracker 232 is secured, irrespective of the specific type or configuration of the localizer 230 and/or tracker 232.

In some versions, the navigation system 202 and/or the localizer 230 could be radio frequency (RF) based. For example, the navigation system 202 may comprise an RF transceiver coupled to the navigation controller 228. Here, certain trackers 232 may comprise markers 252 realized as RF emitters or transponders, which may be passive or may be actively energized. The RF transceiver transmits an RF tracking signal, and the RF emitters respond with RF signals such that tracked states are communicated to (or interpreted by) the navigation controller 228. The RF signals may be of any suitable frequency. The RF transceiver may be positioned at any suitable location to track the objects using RF signals effectively. Furthermore, it will be appreciated that versions of RF-based navigation systems may have structural configurations that are different than the navigation system 202 illustrated throughout the drawings.

In some versions, the navigation system 202 and/or localizer 230 may be electromagnetically (EM) based. For example, the navigation system 202 may comprise an EM transceiver coupled to the navigation controller 228. Here, certain trackers 232 may comprise markers 252 realized as EM components (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 228. The navigation controller 228 may analyze the received EM signals to associate relative states thereto. Here too, it will be appreciated that versions of EM-based navigation systems may have structural configurations that are different than the navigation system 202 illustrated throughout the drawings.

Those having ordinary skill in the art will appreciate that the navigation system 202 and/or localizer 230 may have any other suitable components or structure not specifically recited herein. Furthermore, any of the techniques, methods, and/or components described above with respect to the camera-based navigation system 202 shown throughout the drawings may be implemented or provided for any of the other versions of the navigation system 202 described herein. For example, the navigation system 202 may also be based on one or more of inertial tracking, ultrasonic tracking, image-based optical tracking (e.g., with markers 252 are defined by patterns, shapes, edges, and the like that can be monitored with a camera), or any combination of tracking techniques. Other configurations are contemplated.

With continued reference to FIG. 7, the robotic system 200 may include a robotic arm 256 operatively attached to a support element 258 and configured to maintain alignment of the tool 206 relative to the target site ST. The robotic arm 256 may extend between a base end 260 and a mount end 262 arranged for movement relative to the base end 260. The robotic system 200 may further includes an end effector 264 attached to the mount end 262 of the robotic arm 256 and configured to support one or more types of tools 206, instruments, and the like. More specifically, the robotic system 200 may further include a tool guide 266 supported by the end effector 264, and the tool guide 266 may be configured to support the tool 206 relative to a trajectory that is aligned or otherwise determined relative to the surgical site ST on the patient P.

The robotic arm 256 may comprise a multi-joint arm that includes a plurality of linkages connected by joints having actuator(s) and optional encoder(s) (not shown in detail) to enable the linkages to bend, rotate and/or translate relative to one another in response to control signals from a robot control system. The robotic arm 256 may be fixed to the mobile medical imaging system 100, such as on the support element 258 (e.g. a curved rail) that may extend concentrically over the outer surface of the imaging gantry 104 of the mobile medical imaging system 100 and that may be located close to the target site ST of the patient P. Where the robotic arm is attached to the mobile medical imaging system, such as to the gantry 104, the latching system (304) may retain the gantry mount 106 in the park pose PP as the robotic arm 256 operates to ensure stability of the imaging gantry 104 and the robotic arm 256. In some versions, the robotic arm 256 could be coupled to a mobile cart (not shown) or to another type of support element 258 that is not necessarily coupled to the mobile medical imaging system 100. Although a single robotic arm 256 is shown in FIG. 7, it will be understood that the robotic system 200 may include multiple robotic arms attached to suitable support structure(s). Other configurations are contemplated.

The support element 258 may form a semicircular arc and may be concentric with the outer circumference of the imaging gantry 104. The support element 258 may extend around at least 25%, such as between about 30-50% of the outer circumference of the imaging gantry 104. The support element 258 may extend around at least a portion of the outer circumference of the imaging gantry 104 that is located above the target site ST of the patient P. More specifically, the base end 260 of the robotic arm 256 (e.g., the end of the robotic arm 256 opposite the end effector 264) may be fixed to the support element 258, in a non-limiting example, at a position that is less than about 2 meters, such as less than about 1 meter (e.g., between 0.5 and 1 meter) from the surgical site ST of the patient P during a surgical procedure.

In versions, the support element 258 may extend along a semicircular arc having a radius that is greater than about 33 inches, such as greater than about 35 inches (e.g., between 33 and 50 inches). The support element 258 may be spaced from the outer surface of the imaging gantry 104 by a pre-determined distance, which may be from less than an inch (e.g., 0.5 inches) to 6 or 10 inches or more. In some versions, the support element 258 may be spaced from the imaging gantry 104 by an amount sufficient to enable the tilt motion of the imaging gantry 104 with respect to the gimbal 134 supporting the imaging gantry 104 over at least a limited range of motion. Additionally, in some versions, the support element 258 may comprise one or more straight segments (e.g., rail segments), where at least a portion of the support element 258 may extend over the top surface of the imaging gantry 104. Other configurations are contemplated.

A carriage 270 may be located on the support element 258 and may include a mounting surface 272 for mounting the base end 260 of the robotic arm 256 to the carriage 270. As shown in FIG. 7, the carriage 270 may extend from the support element 258 towards a first (e.g., front) face of the imaging gantry 104. The mounting surface 272 for the robotic arm 256 may extend beyond the first (e.g., front) face of the imaging gantry 104 and the robotic arm 256 may extend over the first (e.g., front) face of the imaging gantry 104. In some versions, the configuration of the carriage 270 and the mounting surface 272 may be reversed such that the mounting surface 272 extends beyond the second (e.g., rear) face of the imaging gantry 104, and the robotic arm 256 may extend over the second (e.g., rear) face of the imaging gantry 104. In this configuration, the patient support 117 may be configured such that the patient support 117 and patient P extend into or through the imaging bore 114, and a portion of the patient P requiring surgical intervention (e.g., the cranium) may be accessed from the second (e.g., rear) side of the imaging gantry 104.

In some versions, the carriage 270 and the robotic arm 256 attached thereto may be moved to different positions along the length of support element 258 (e.g., any arbitrary position between a first end 276 and a second end 278 of the support element 258). The carriage 270 and the robotic arm 256 may be fixed in place at a particular desired position along the length of the support element 258. In some versions, the carriage 270 may be moved manually (e.g., positioned by an operator at a particular location along the length of the support element 258 and then clamped or otherwise fastened in place). Alternately, the carriage 270 may be driven to different positions using a suitable drive mechanism (e.g., a motorized belt drive, friction wheel, gear tooth assembly, cable-pulley system, etc., not shown in detail). The drive mechanism may be located on the carriage 270 and/or the support element 258, for example. An encoder mechanism may be utilized to indicate the position of the carriage 270 and the base end 260 of the robotic arm 256 on the support element 258. Although the version of FIG. 7 illustrates one robotic arm 256 mounted to the support element 258, it will be understood that more than one robotic arm 256 may be mounted to the support element 258 via respective carriages 270.

In some versions, the robotic arm 256 may be mounted directly to the support element 258, such as on a mounting surface 272 that is integrally formed on the support element 258. In such an version, the position of robotic arm 256 may not be movable along the length of the support element 258. In other versions, the robotic arm 256 may be secured to any other portion of the mobile medical imaging system 100, such as directly mounted to the imaging gantry 104. Alternatively, the robotic arm 156 may be mounted to the patient support 117 or pedestal 116, to any of the wall, ceiling or floor in the operating room, or to a separate cart as noted above. In some versions, the robotic arm 256 may be mounted to a separate mobile shuttle, similar to as is described in U.S. Pat. No. 11,103,990, entitled “System and Method for Mounting a Robotic Arm in a Surgical Robotic System,” the disclosure of which is hereby incorporated by reference in its entirety. Although a single robotic arm 256 is shown in FIG. 7, it will be understood that two or more robotic arms 256 may be utilized.

Those having ordinary skill in the art will appreciate that the robotic arm 256 can be employed to aid in the performance of various types of surgical procedures, such as a minimally-invasive spinal surgical procedure or various other types of orthopedic, neurological, cardiothoracic and general surgical procedures. In the version of FIG. 7, the robotic arm 256 may be used to assist a surgeon performing a surgical procedure in the lumbar spinal region of a patient. The robotic arm 256 may also be used for thoracic and/or cervical spinal procedures. The procedures may be performed posteriorly, anteriorly or laterally. Other configurations are contemplated.

In some versions, the robotic arm 256 may be controlled to move the end effector 264 to one or more pre-determined positions and/or orientations with respect to a patient P, such as to and/or along a trajectory defined relative to the anatomy of the patient P. As discussed above, the end effector 264 may be realized as or may otherwise support various types of instruments and/or tools 206 including, but not limited to, a needle, a cannula, a dilator, a cutting or gripping instrument, a scalpel, a drill, a screw, a screwdriver, an electrode, an endoscope, an implant, a radiation source, a drug, etc., that may be inserted into the body of the patient P. In some versions, the end effector 264 may be realized as a hollow tube or cannula configured to receive a surgical tool 206, including without limitation a needle, a cannula, a dilator, a cutting or gripping instrument, a scalpel, a drill, a screw, a screwdriver, an electrode, an endoscope, an implant, a radiation source, a drug, and the like. The surgical tool 206 may be inserted into or otherwise adjacent to the patient's body through the hollow tube or cannula by a surgeon. The robotic arm 256 may be controlled to maintain the position and orientation of the end effector 264 with respect to the patient P to ensure that the surgical tool(s) 206 follow a desired trajectory through the patient's body to reach the target site ST. The target site ST may be determined preoperatively and/or intraoperatively, such as during a surgical planning process, based on patient images which may be obtained using the mobile medical imaging system 100.

In the representative version illustrated herein, the navigation system 202 tracks the robotic arm 256 within the localizer coordinate system LCLZ via the robot tracker 232R. To this end, a control loop may continuously read the tracking data and current parameters (e.g., joint parameters) of the robotic arm 256, and may send instructions to the navigation controller 228 and/or to the system controller 144 (and/or some other controller, such as a robot controller) to cause the robotic arm 256 to move to a desired position and orientation within the localizer coordinate system LCLZ.

In some versions, a surgeon may use one or more portions of the robotic system 200 as a planning tool for a surgical procedure, such as by setting trajectories within the patient for inserting tools 206, as well as by selecting one or more target sites ST for a surgical intervention within the patient's body. The trajectories and/or target sites ST set by the surgeon may be saved (e.g., in a memory of a computer device) for later use during surgery. In some versions, the surgeon may be able to select stored trajectories and/or target sites ST using the robotic system 200, and the robotic arm 256 may be controlled to perform a particular movement based on the selected trajectory and/or target site ST. For example, the robotic arm 256 may be moved to position the end effector 264 of the robotic arm 256 into alignment with the pre-defined trajectory and/or over the pre-determined target site ST. As discussed above, the end effector 264 may include the tool guide 266 which may be used to guide the tool 206 relative to the patient's body along the pre-defined trajectory and/or to the pre-defined target site ST.

As discussed above, the localizer 230 may include a camera unit 248 with one or more optical position sensors 250. More specifically, the optical position sensors 250 may be light sensors capable of sensing changes in infrared (IR) emitted within a field of view. In some versions, the localizer 230 may include one or more radiation sources (e.g., one or more diode rings) that direct radiation (e.g., IR radiation) into the surgical field, where the radiation may be reflected by the markers 252 and received by the cameras. In the illustrated version, certain active markers 252 (e.g., active markers 252 which define the robot tracker 232R) are configured to emit IR light detectable by the optical position sensors 250 of the localizer 230. The navigation controller 228 may be coupled to the localizer 230 and may determine the positions and/or orientations of markers 252 detected by the optical position sensors 250 using, for example, triangulation and/or transformation techniques. A 3D model and/or mathematical simulation of the surgical space may be generated and continually updated using motion tracking software implemented by the navigation controller 228.

Additionally, the patient tracker 232A may be rigidly attached to a portion of the patient's anatomy in the anatomical region of interest adjacent to the target site ST (e.g., clamped or otherwise attached to the ilium, to the spinous process of the vertebrae, and the like) to enable the anatomical region of interest to be continually tracked by the navigation system 202. In the illustrated version, the robot tracker 232R is rigidly attached to the end effector 264 of the robotic arm 256 to enable the robotic arm 256 to be tracked using the navigation system 202. Using the pose of the end effector tracker 282 (as well as of the patient tracker 232) monitored within the localizer coordinate system LCLZ by the localizer 230, the navigation controller 228 and/or some other controller (e.g., a robot controller) may include software configured to perform transformations between joint coordinates of the robotic arm 256 and the localizer coordinate system LCLZ which, in turned, may be utilized by the robotic arm 256 to control or otherwise adjust the position and/or orientation of the end effector 264 with respect to the patient P. In some versions, the robotic arm 256 may include multiple robot trackers 232R and/or robot trackers 232R other than the end effector tracker 282 (e.g., on joints of the arm). Other configurations are contemplated.

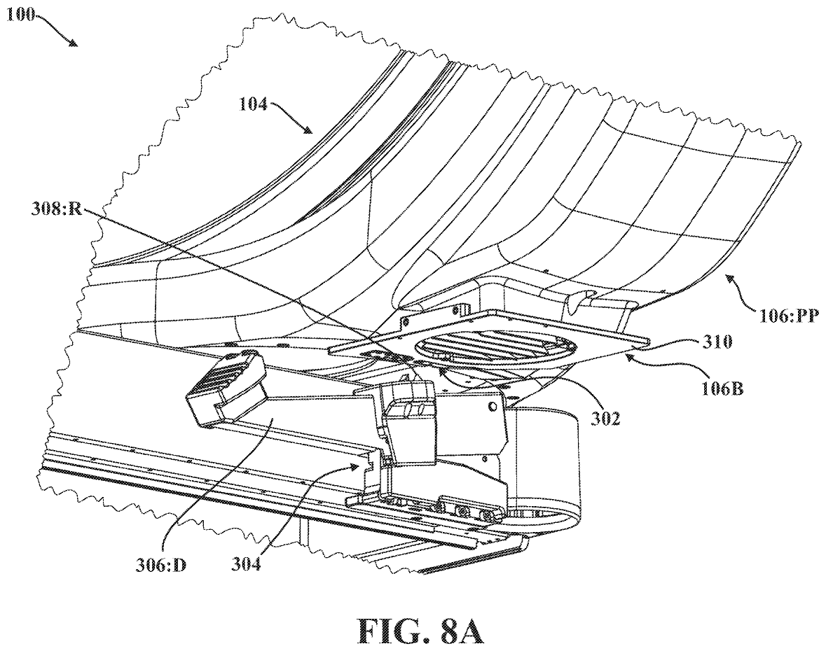

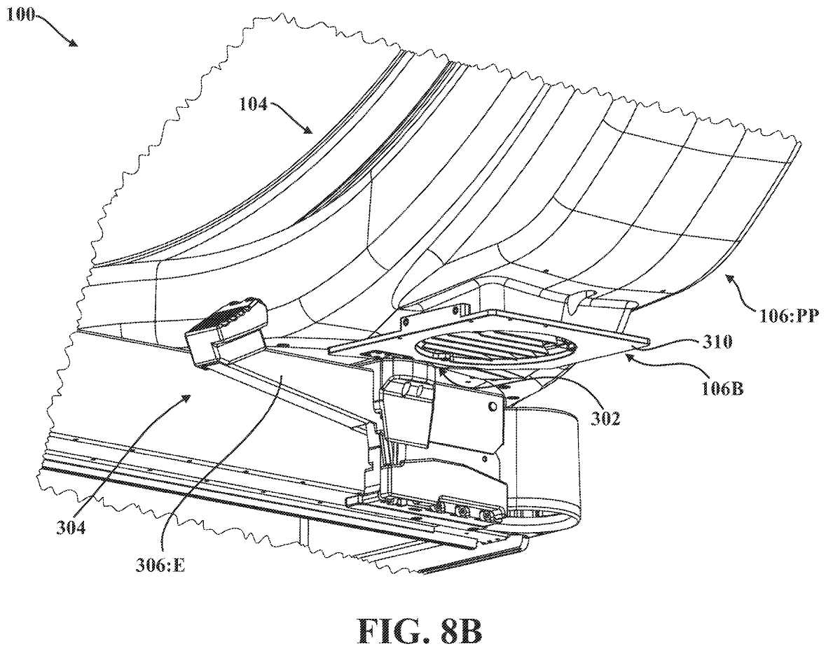

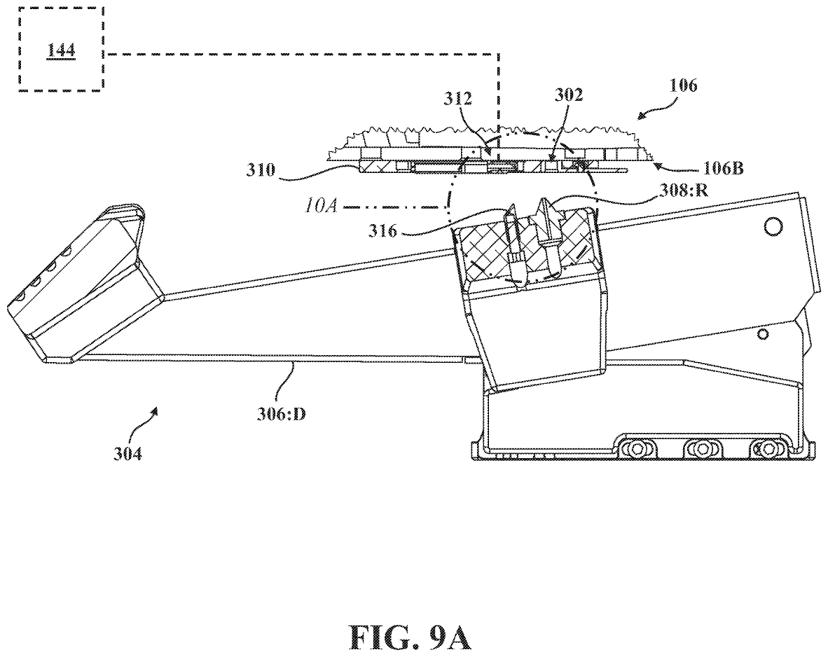

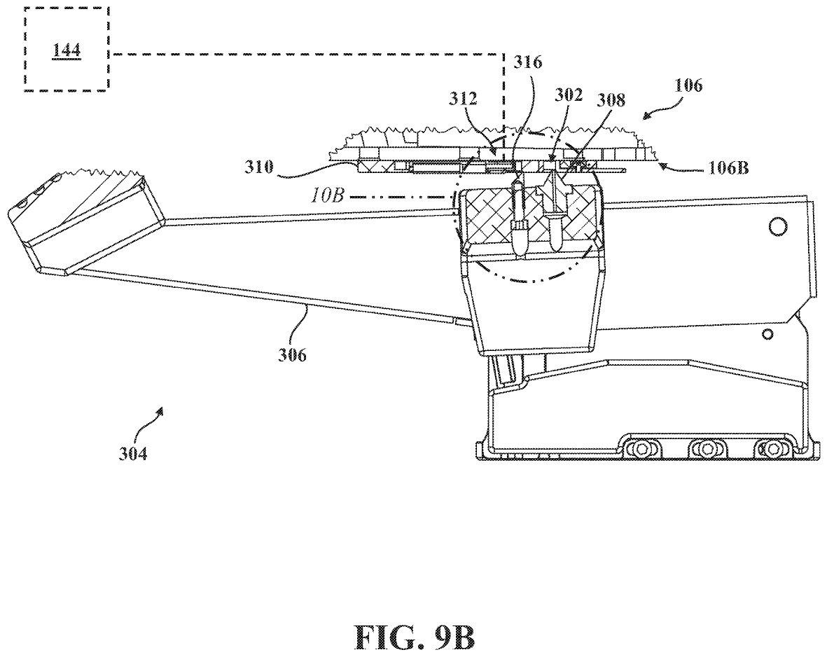

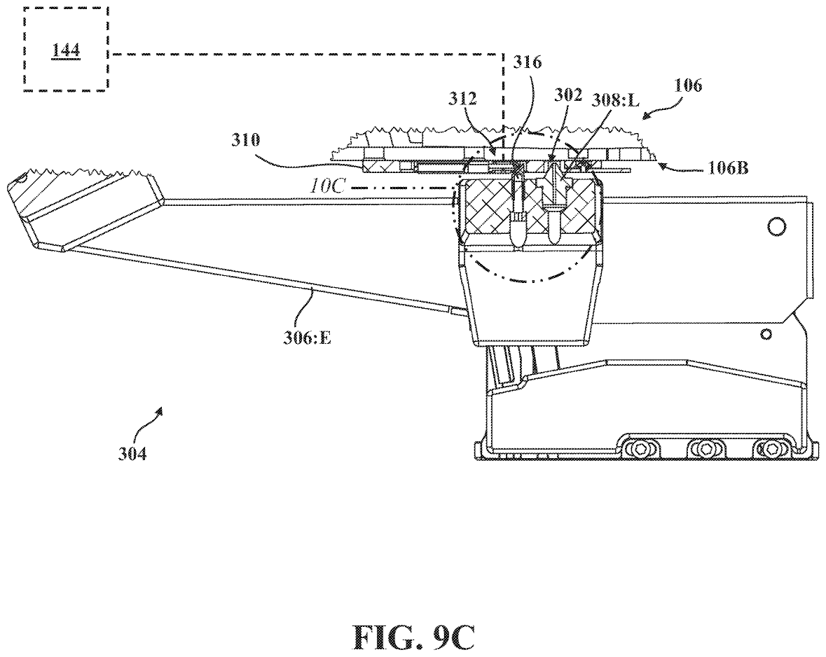

Referring to FIG. 9A, the mobile medical imaging system 100 also includes a catch 302 operatively attached to the gantry mount 106 for concurrent movement with the gantry mount 106 between the plurality of track poses. Referring to FIGS. 8A and 8B, in some examples, the catch 302 is defined by a bottom portion 106B of the gantry mount 106. In the illustrated example, the bottom portion 106B of the gantry mount 106 includes a catch plate 310 defining the catch 302. However, it should be appreciated that other suitable configurations for operatively attaching the catch 302 to the gantry mount 106 are contemplated.

Referring to FIGS. 1A-1B and 8A-10C, the mobile medical imaging system 100 also includes a latching system 304. The latching system 304 includes a pedal 306 operatively attached to the base 102. The pedal 306 supports a latch 308 (best shown in FIGS. 8A and 9A-10C) and is configured for movement between an engaged position 306E and a disengaged position 306D. FIGS. 1A, 8B, 9B and 10B show the pedal in the engaged position 306E. Where the pedal 306 is placed in the engaged position 306E, the pedal 306 places the latch 308 in a lock position 308L with the latch 308 engaging the catch 302 to retain the imaging gantry 104 in the park pose PP. FIGS. 1B, 8A, and 9C show the pedal in the disengage position 306D. Where the pedal is in the disengaged position 306D, the pedal 306 places the latch 308 in a released position 308R with the latch 308 spaced from the catch 302 to permit translation of the gantry mount 106 away from the park pose PP in response to one of powered operation of the translation mechanism 128, and user-applied force applied to back-drive the translation mechanism 128 (i.e., user-applied force to translate the gantry mount 106 along the track 108).

With continued reference to FIGS. 1A and 1B, as described above, in some examples the gantry mount member 132 may be configured to rotate relative to the base 102 about the first axis FA. In these examples, the latching system 304 may be configured to engage the catch 302 to prevent such rotation of the gantry mount 106 about the first axis FA. To this end, the latch 308 and the catch 302 may be spaced from the first axis FA (as shown in FIGS. 1A and 1B) such that the latching system 304 also inhibits rotation of the gantry mount 106 (and, consequently, of the imaging gantry 104) relative to the base 102 where the latch 308 is in the lock position 308L and the gantry mount 106 is in the park pose PP, and the latching system 304 permits rotation of the gantry mount 106 (and, consequently, of the imaging gantry 104) relative to the base 102 where the latch is in the released position 308R.

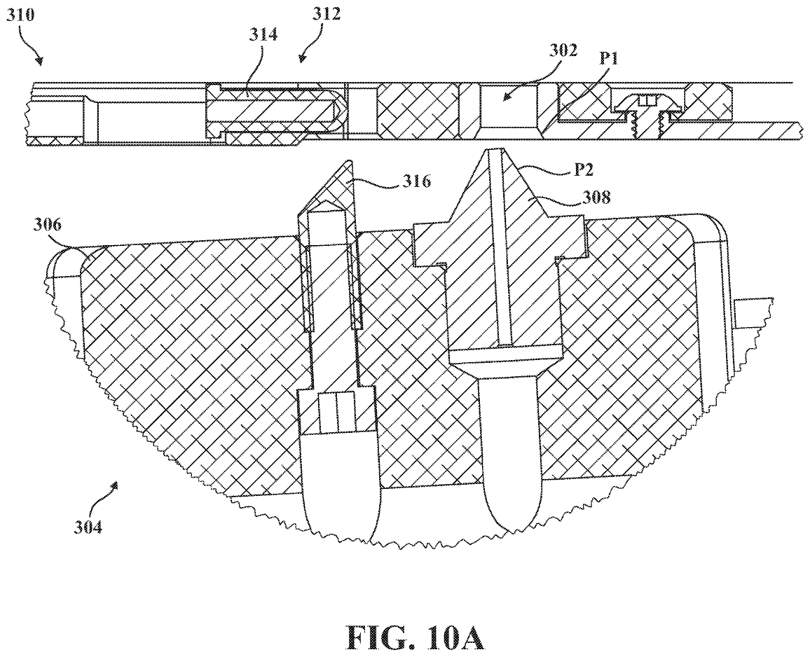

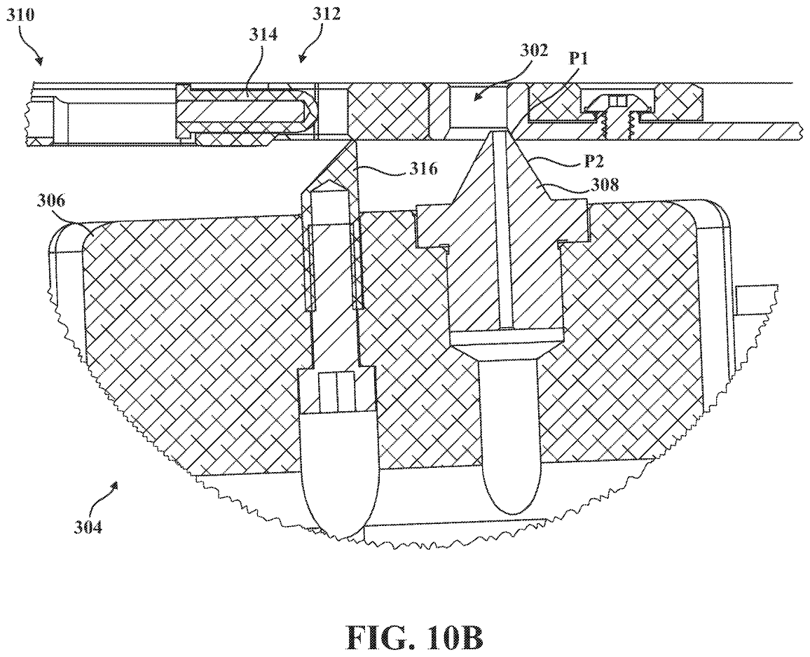

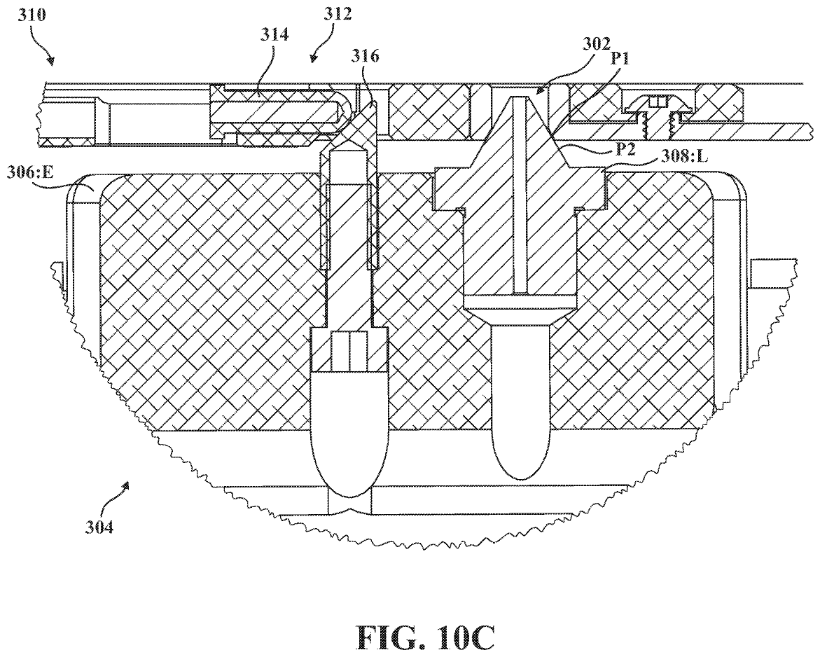

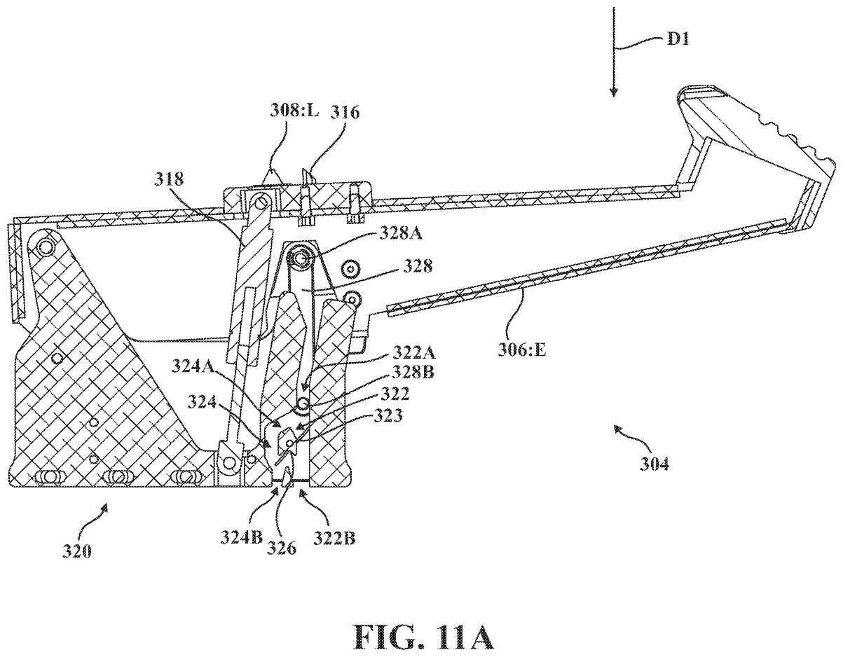

As best shown in FIGS. 9A-10C, in some examples, the catch 302 may define a first profile P1, and the latch 308 may define a second profile P2 shaped for engagement with the first profile P1 to urge the gantry mount 106 along the track 108 to align the latch 308 with the catch 302 in the lock position 302L. For example, FIG. 9A shows a cross-sectional representation of the pedal 306 relative to the catch 302 with the pedal 306 in the disengaged position 306D such that the latch 308 is spaced from the catch 302 to permit translation of the gantry mount 106 away from the park pose PP, and FIG. 10A shows a detail view of the latch 308 spaced from the catch 302. FIGS. 9B and 10B show the latch 308 engaging the catch 302 as the pedal 306 moves from the disengaged position 306D to the engaged position 306E. Notably, in the examples illustrated in the sequence between FIGS. 9A and 10A to FIGS. 9B and 10B, respectively, the latch 308 and the catch 302 are not perfectly aligned. However, as shown in FIGS. 9B and 10B, the second profile P2 of the latch 308 may cooperate with the first profile P1 of the catch 302 in order to urge the gantry mount 106 along the track 108 toward the park pose PP. Accordingly, FIGS. 9C and 10C show the pedal 306 in the engaged position 306E with the latch 308 engaged with the catch 302 to retain the gantry mount 106 in the park pose PP.

With continued reference to FIGS. 9A-10C, in some examples, the gantry mount 106 may further include a catch sensor 312 arranged adjacent to the catch 302. In some examples, the catch sensor 312 is coupled to the catch plate 310. The catch sensor 312 may be in communication with the system controller 144 and configured to generate a catch engagement signal in response to engagement of the latch 308 with the catch 302 in the lock position 308L. In response to the catch engagement signal indicating that the pedal 306 is in the engaged position 306E and the latch 308 is in the lock position 308L, the system controller 144 may be configured to inhibit operation of the translation motor 138 and/or the gantry motor 140 to inhibit translation and/or rotation of the gantry mount 106 relative to the base 102.

As best shown in FIGS. 10A-10C, in some examples, catch sensor 312 may be configured to generate the catch engagement signal based on displacement of a catch sensor member 314. For example, the catch sensor 312 may be a hall-effect sensor or any other suitable sensor for generating the catch engagement signal. Accordingly, the latching system 304 may further include a sensor projection 316 disposed on the pedal 306 and adjacent to the latch 308. The sensor projection 316 may be configured to engage the catch sensor member 314 of the catch sensor 312 when the latch 308 is in the lock position 308L (best shown in FIG. 10C) such that the catch sensor member 314 displaces and the catch sensor 312 generates the catch engagement signal. In some examples, such as best shown in FIGS. 10A-10C, the sensor projection 316 may define a tapered profile such that the catch sensor member is not sufficiently displaced to generate the catch engagement signal until the latch 308 reaches the lock position 308L.

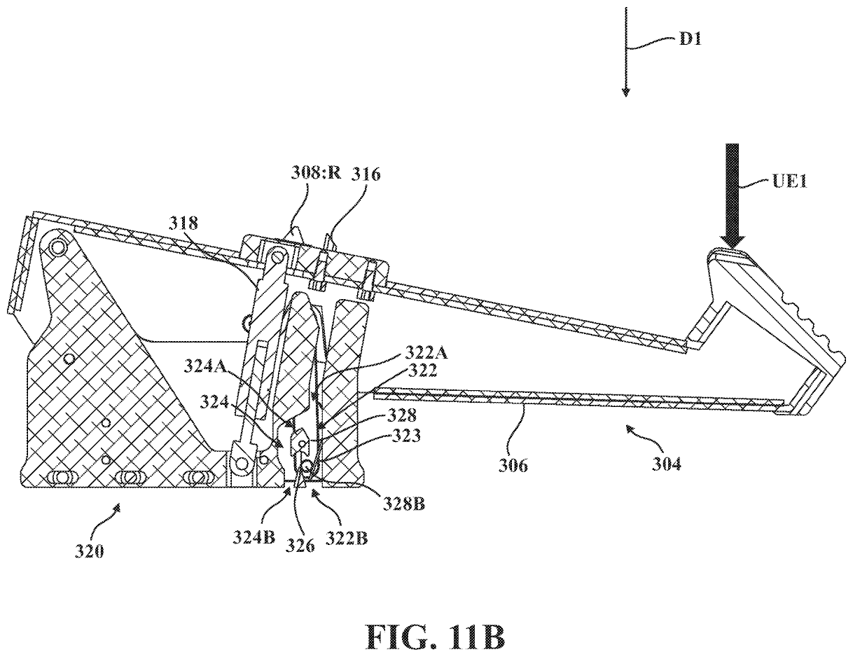

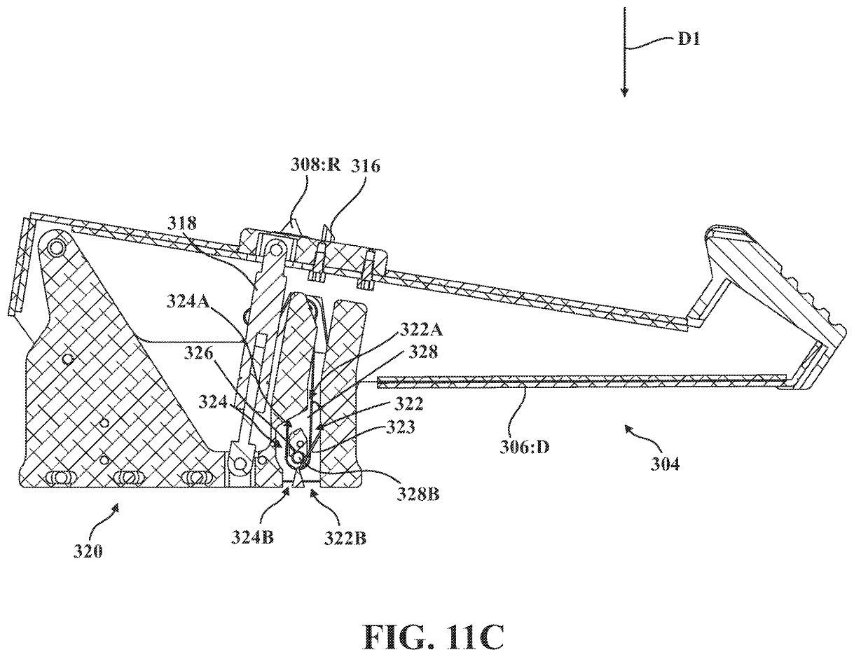

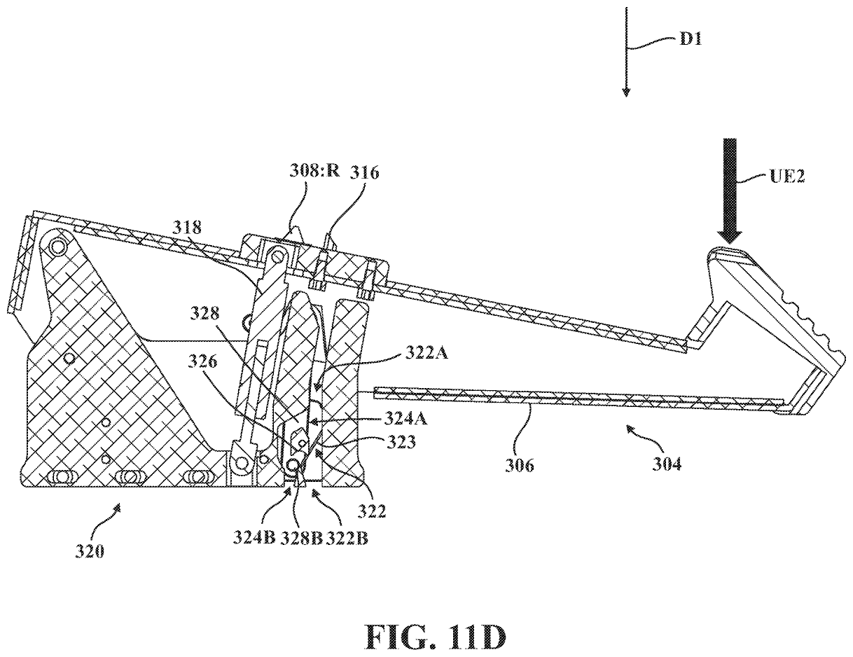

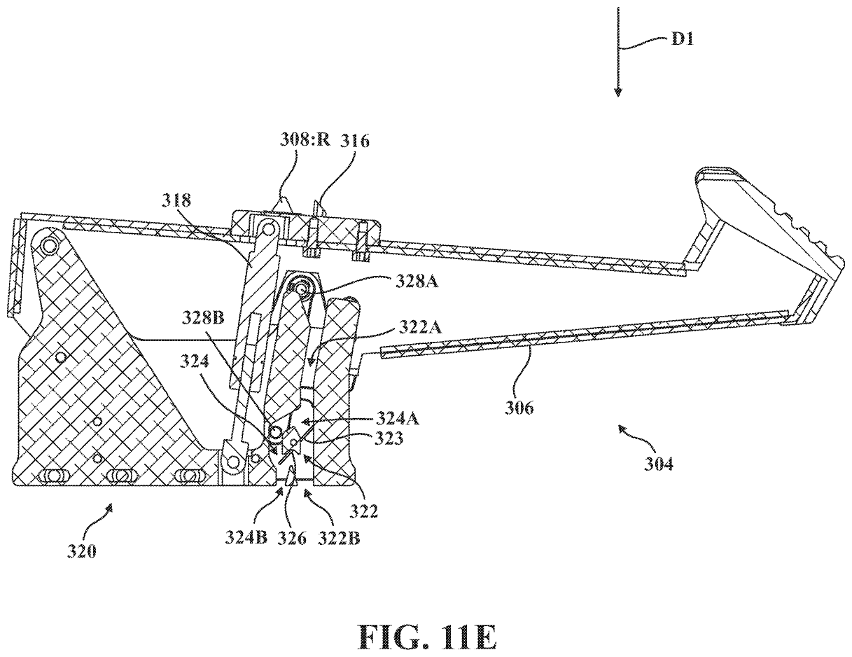

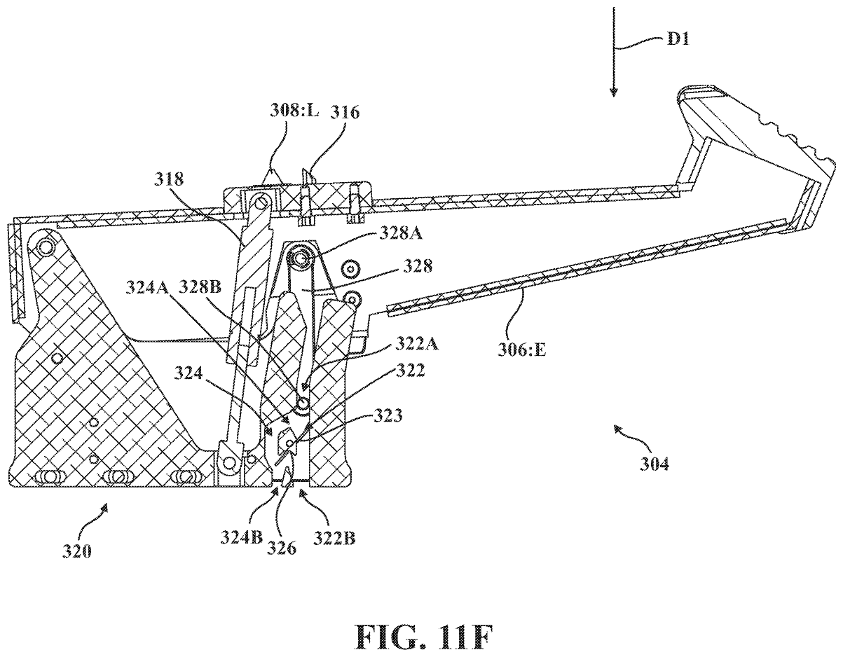

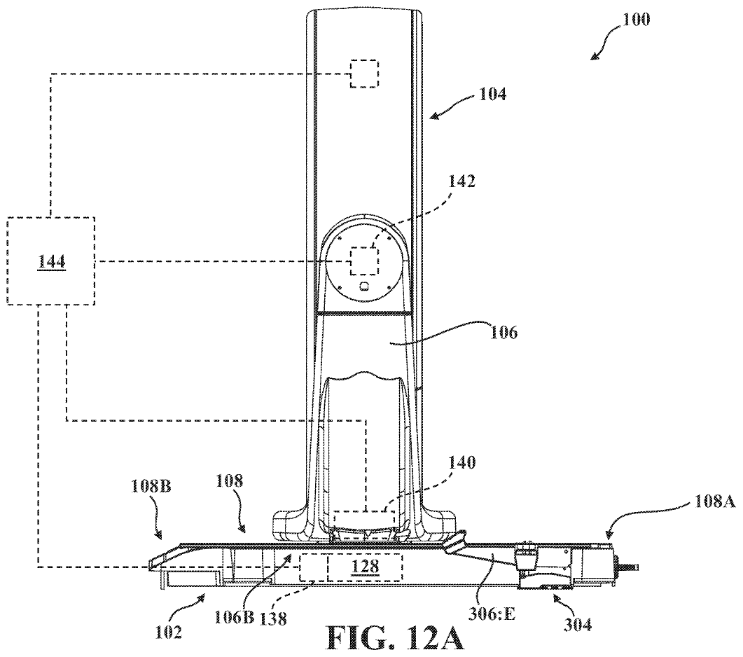

In some examples, the latching system 304 includes a biasing member 318 operatively attached to the pedal 306 to bias the pedal 306 to the engaged position 306D. FIGS. 11A-11F shown cross-sectional representations of the latching system 304 taken along the biasing member 318 to reveal the internal componentry of the latching system 304 (described in further detail below). Referring to FIGS. 12A-12C, in some examples, the pedal 306 is disposed above the bottom portion 106B of the gantry mount 106 when the pedal 306 is in the engaged position 306E (shown in FIGS. 12A and 12C), and the pedal 306 is disposed below the bottom portion 106B of the gantry mount 106 when the pedal 306 is in the disengaged position 306D (shown in FIG. 12B). In some examples, the bottom portion 106B of the gantry mount 106 is configured to deflect the pedal 306 from the engaged position 306E toward the disengaged position 306D as the gantry mount 106 translates along the track 108 from the second track end 108B toward the first track end 108A. FIG. 12A shows the gantry mount 106 arranged between the first track end 108A and the second track end 108B of the track 108. FIG. 12B shows the gantry mount 106 translating along the track 108 toward the first track end 108A and the bottom portion 106B of the gantry mount 106 abutting the pedal 306 to deflect the pedal 306 from the engaged position 306E toward the disengaged position 306D. FIG. 12C shows the gantry mount 106 reaching the first track end 108A of the track 108 and the pedal 306 returning to the engaged position 306E due to the biasing member 318. In some examples, due to the biasing member 318, the latch 308 may be configured to automatically engage the catch 302 in response to the gantry mount 106 reaching the park pose PP.

Referring back to FIGS. 11A-11F, in some examples, the latching system 304 may be configured as a “push-push” system. In other words, the pedal 306 may only be arranged for user engagement in a singular direction (i.e., a first direction D1) and the pedal 306 may behave differently based on whether the engagement is a first user engagement or a second, subsequent, user engagement in the first direction D1, as described in the following passages.

FIG. 11A shows the pedal 306 in the engaged position 306E. Referring to FIG. 11B, the pedal 306 is arranged for a first user engagement (generally indicated with an arrow and reference number UE1) in the first direction D1. In response to the first user engagement UE1, the pedal 306 is configured to move from the engaged position 306E toward the disengage position 306D to disengage the latch 308 from the catch 302. Referring to FIG. 11C, the latching system 304 is configured to retain the pedal 306 in the disengaged position 306D after the pedal 306 moves toward the disengaged position 306D in response to the first user engagement UE1. Referring to FIGS. 11D-11F, the latching system 304 is configured to release the pedal 306 from the disengaged position 306D in response to the second user engagement (generally indicated with an arrow and reference number UE2) in the first direction D1. As a result, the biasing member 318 urges the pedal 306 back to the engaged position 306E such the latch 308 may engage the catch 302 to retain the gantry mount 106 in the park pose PP.

One example of a structure enabling the push-push function of the latch system 304 described above is shown in FIGS. 11A-11F. In this example, the latching system 304 includes a housing 320. The housing 320 may support the pedal 306 for pivoting movement between the engaged position 306E and the disengaged position 306D. The housing 320 may also define a first channel 322 and a second channel 324. The first channel 322 may extend between a first top end 322A and a first bottom end 322B. The first bottom end 322B may be arranged adjacent to a link landing 326. The second channel 324 may extend between a second bottom end 324B arranged adjacent to the link landing 326 and a second top end 324A connected to the first channel 322. Accordingly, the first channel 322, the second channel 324, and the link landing 326 may define a loop that a link 328 travels about during operation of the pedal 306. The link 328 may extend between a first end 328A operatively attached to the pedal 306 and a second end 328B. The second end 328B of the link 328 travels along the first channel 322 in response to the first user engagement UE1 (shown sequentially in FIGS. 11A-11B). Referring to FIG. 11C, the latching system 304 may further include a link biasing member 323 configured to urge the second end 328B of the link 328 toward the link landing 326 as the second end 328B reaches the first bottom end 322B in response to the first user engagement UE1 such that the second end 328B abuts the link landing 326 to retain the pedal 306 in the disengaged position 306D. Referring to FIG. 11D, in response to the second user engagement UE2, the link biasing member 323 is configured to urge the second end 328B of the link 328 into the second channel 324 such that the second end 328B of the link 328 travels along the second channel 324 to release the pedal 306 from the disengaged position (shown in FIGS. 11D and 11E). Ultimately, referring to the sequence between FIGS. 11E and 11F, once the second end 328B of the link 328 reaches the second top end 324A of the second channel 324, the second end 328B returns to the first channel 322 and the biasing member 318 biases the pedal 306 back to the engaged position 306E (shown in FIG. 11F). Other suitable structures for enabling the push-push function of the latch system 304 are contemplated.

It will be further appreciated that the terms “include,” “includes,” and “including” have the same meaning as the terms “comprise,” “comprises,” and “comprising.”

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The present disclosure also comprises the following clauses, with specific features laid out in dependent clauses, that may specifically be implemented as described in greater detail with reference to the configurations and drawings above.

### Clauses

I. A mobile medical imaging system comprising:
a base defining a track extending between a first track end and a second track end;
an imaging gantry having at least one imaging component and defining an imaging bore;
a gantry mount supporting the imaging gantry for movement along the track between a plurality of track poses including a park pose defined with the gantry mount arranged adjacent to the first track end;
a translation mechanism interposed between the base and the gantry mount to drive the gantry mount between the plurality of track poses in an imaging mode to acquire image data of a patient within the imaging bore;
a catch operatively attached to the gantry mount for concurrent movement between the plurality of track poses; and
a latching system including a pedal operatively attached to the base and supporting a latch, the pedal configured for movement between:
   an engaged position that places the latch in a lock position with the latch engaging the catch to retain the imaging gantry in the park pose, and
   a disengaged position that places the latch in a released position with the latch spaced from the catch to permit translation of the gantry mount away from the park pose in response to one of: powered operation of the translation mechanism, and user-applied force applied to back-drive the translation mechanism.

II. The mobile medical imaging system of clause I, wherein the gantry mount includes:
a gantry mount base operatively attached to the base,
a gantry mount member operatively attached to the gantry mount base for rotation relative to the gantry mount base, the gantry mount member supporting the imaging gantry such that the gantry mount member and the imaging gantry are configured to rotate together about a first axis relative to the base.

III. The mobile medical imaging system of clause II, wherein:
the latch and the catch are spaced from the first axis,
the latching system inhibits rotation of the gantry mount and the imaging gantry relative to the base where the latch is in the lock position and the gantry mount is in the park pose, and
the latching system permits rotation of the gantry mount and the imaging gantry relative to the base where the latch is in the released position.

IV. The mobile medical imaging system of any of clauses II-III, wherein:
the imaging bore defines an imaging axis that is parallel to the track where the gantry mount is in the park pose and the mobile medical imaging system is in the imaging mode, and
the plurality of track poses of the gantry mount includes a transport pose where the gantry mount is arranged between the first track end and the second track end and the gantry mount member and the imaging gantry are rotated such that the imaging axis is transverse to the track.

V. The mobile medical imaging system of any of clauses II-IV, wherein the gantry mount member includes a gimbal having a pair of arms, each arm coupled to an opposite side of the imaging gantry to support the imaging gantry above the base and the gimbal, wherein the imaging gantry is configured to tilt about a second axis relative to the gimbal.

VI. The mobile medical imaging system of any of clauses II-V, further comprising:
a gantry motor interposed between the gantry mount base and the gantry mount member for rotating the gantry mount member relative to the base about the first axis; and
a controller in communication with the gantry motor to control operation of the gantry motor.

VII. The mobile medical imaging system of clause VI, wherein the gantry mount further comprises a catch sensor in communication with the controller and arranged adjacent to the catch and configured to generate a catch engagement signal in response to engagement of the latch with the catch; and
wherein operation of the gantry motor is inhibited based on the catch engagement signal indicating that the pedal is in the engaged position.

VIII. The mobile medical imaging system of clause VII, wherein the latching system further includes a sensor projection disposed adjacent to the latch and configured to engage the catch sensor when the latch is in the lock position such that the catch sensor generates the catch engagement signal.

IX. The mobile medical imaging system of any of clauses I-VIII, further comprising:
a translation motor operatively attached to the translation mechanism to drive the gantry mount between the plurality of track poses; and
a controller in communication with the translation motor to control operation of the translation motor.

X. The mobile medical imaging system of clause IX, wherein the gantry mount further comprises a catch sensor in communication with the controller and arranged adjacent to the catch and configured to generate a catch engagement signal in response to engagement of the latch with the catch; and
wherein operation of the translation motor is inhibited based on the catch engagement signal indicating that the pedal is in the engaged position.

XI. The mobile medical imaging system of clause X, wherein the latching system further includes a sensor projection disposed adjacent to the latch and configured to engage the catch sensor when the latch is in the lock position such that the catch sensor generates the catch engagement signal.

XII. The mobile medical imaging system of any of clauses I-XI, wherein the pedal is spaced below the gantry mount when pedal is in the disengaged position.

XIII. The mobile medical imaging system of any of clauses I-XII, wherein the catch is defined by a bottom portion of the gantry mount, and the latch moves toward the bottom portion of the gantry mount as the latch moves between the released position and the lock position.

XIV. The mobile medical imaging system of clause XIII, wherein:
the catch defines a first profile, and
the latch defines a second profile shaped for engagement with the first profile to urge the gantry mount along the track to align the latch with the catch in the lock position.

XV. The mobile medical imaging system of any of clauses I-XIV, wherein latching system further comprises a biasing member operatively attached to the pedal to bias the pedal toward the engaged position.

XVI. The mobile medical imaging system of clause XV, wherein the pedal is disposed above a bottom portion of the gantry mount when the pedal is in the engaged position, and the bottom portion of the gantry mount is configured to deflect the pedal from the engaged position toward the disengaged position as the gantry mount translates from the second track end toward the first track end.

XVII. The mobile medical imaging system of any of clauses XV-XVI, wherein the pedal is arranged for user engagement in a first direction and the latching system is configured to retain the pedal in the disengaged position in response to a first user engagement with the pedal in the first direction for disengaging the latch.

XVIII. The mobile medical imaging system of clause XVII, wherein the latching system is configured to release the pedal from the disengaged position in response to a second user engagement with the pedal in the first direction for engaging the latch.

XIX. The mobile medical imaging system of clause XVIII, wherein the latching system further comprises:
a housing defining a first channel extending between a first top end and a first bottom end, a link landing arranged adjacent to the first bottom end, and a second channel extending between a second bottom end arranged adjacent to the link landing, and a second top end connected to the first channel; and
a link extending between a first end operatively attached to the pedal and a second end, wherein the second end of the link travels along the first channel in response to the first user engagement to abut the link landing to retain the pedal in the disengaged position, and wherein the second end of the link travels along the second channel in response to the second user engagement to release the pedal from the disengaged position.

XX. The mobile medical imaging system of any of clauses I-XIX, wherein the at least one imaging component comprises a rotor supporting an x-ray source and a detector and disposed within a housing defined by the imaging gantry for rotation around the imaging bore.

XXI. The mobile medical imaging system of clause XX, wherein the x-ray source includes a fan-beam x-ray source, and the detector includes an array of detectors.

XXII. The mobile medical imaging system of clause XXI, wherein the rotor rotates around the imaging bore as the translation mechanism drives the gantry mount along the track in the imaging mode to acquire helical scan x-ray CT images of a patient within the imaging bore.

XXIII. The mobile medical imaging system of any of clauses I-XXII, further comprising a pedestal mounted to the base adjacent to the first track end and configured to support a patient support above the base.

XXIV. The mobile medical imaging system of any of clauses I-XXIII, further comprising a robotic arm extending between a base end operatively attached to the imaging gantry and a mount end arranged for movement relative to the base end.

XXV. The mobile medical imaging system of clause XXIV, further comprising an end effector attached to the mount end of the robotic arm and configured to support a tool for engaging a target site.

XXVI. The mobile medical imaging system of clause XXV, wherein the robotic arm is configured to maintain alignment of the tool relative to the target site.

1. A mobile medical imaging system comprising:
a base defining a track extending between a first track end and a second track end;
an imaging gantry having at least one imaging component and defining an imaging bore;
a gantry mount supporting the imaging gantry for movement along the track between a plurality of track poses including a park pose defined with the gantry mount arranged adjacent to the first track end;
a translation mechanism interposed between the base and the gantry mount to drive the gantry mount between the plurality of track poses in an imaging mode to acquire image data of a patient within the imaging bore;
a catch operatively attached to the gantry mount for concurrent movement between the plurality of track poses; and
a latching system including a pedal operatively attached to the base and supporting a latch, the pedal configured for movement between:
an engaged position that places the latch in a lock position with the latch engaging the catch to retain the imaging gantry in the park pose, and
a disengaged position that places the latch in a released position with the latch spaced from the catch to permit translation of the gantry mount away from the park pose in response to one of: powered operation of the translation mechanism, and user-applied force applied to back-drive the translation mechanism.

2. The mobile medical imaging system of claim 1, wherein the gantry mount includes:
a gantry mount base operatively attached to the base,
a gantry mount member operatively attached to the gantry mount base for rotation relative to the gantry mount base, the gantry mount member supporting the imaging gantry such that the gantry mount member and the imaging gantry are configured to rotate together about a first axis relative to the base.

3. The mobile medical imaging system of claim 2, wherein:
the latch and the catch are spaced from the first axis,
the latching system inhibits rotation of the gantry mount and the imaging gantry relative to the base where the latch is in the lock position and the gantry mount is in the park pose, and
the latching system permits rotation of the gantry mount and the imaging gantry relative to the base where the latch is in the released position.

4. The mobile medical imaging system of claim 2, wherein:
the imaging bore defines an imaging axis that is parallel to the track where the gantry mount is in the park pose and the mobile medical imaging system is in the imaging mode, and
the plurality of track poses of the gantry mount includes a transport pose where the gantry mount is arranged between the first track end and the second track end and the gantry mount member and the imaging gantry are rotated such that the imaging axis is transverse to the track.

5. The mobile medical imaging system of claim 2, wherein the gantry mount member includes a gimbal having a pair of arms, each arm coupled to an opposite side of the imaging gantry to support the imaging gantry above the base and the gimbal, wherein the imaging gantry is configured to tilt about a second axis relative to the gimbal.

6. The mobile medical imaging system of claim 2, further comprising:
a gantry motor interposed between the gantry mount base and the gantry mount member for rotating the gantry mount member relative to the base about the first axis; and
a controller in communication with the gantry motor to control operation of the gantry motor.

7. The mobile medical imaging system of claim 6, wherein the gantry mount further comprises a catch sensor in communication with the controller and arranged adjacent to the catch and configured to generate a catch engagement signal in response to engagement of the latch with the catch;
wherein operation of the gantry motor is inhibited based on the catch engagement signal indicating that the pedal is in the engaged position; and
wherein the latching system further includes a sensor projection disposed adjacent to the latch and configured to engage the catch sensor when the latch is in the lock position such that the catch sensor generates the catch engagement signal.

8. (canceled)

9. The mobile medical imaging system of claim 1, further comprising:
a translation motor operatively attached to the translation mechanism to drive the gantry mount between the plurality of track poses; and
a controller in communication with the translation motor to control operation of the translation motor.

10. The mobile medical imaging system of claim 9, wherein the gantry mount further comprises a catch sensor in communication with the controller and arranged adjacent to the catch and configured to generate a catch engagement signal in response to engagement of the latch with the catch;
wherein operation of the translation motor is inhibited based on the catch engagement signal indicating that the pedal is in the engaged position; and
wherein the latching system further includes a sensor projection disposed adjacent to the latch and configured to engage the catch sensor when the latch is in the lock position such that the catch sensor generates the catch engagement signal.

11. (canceled)

12. The mobile medical imaging system of claim 1, wherein the pedal is spaced below the gantry mount when pedal is in the disengaged position.

13. The mobile medical imaging system of claim 1, wherein the catch is defined by a bottom portion of the gantry mount, and the latch moves toward the bottom portion of the gantry mount as the latch moves between the released position and the lock position.

14. The mobile medical imaging system of claim 13, wherein:
the catch defines a first profile, and
the latch defines a second profile shaped for engagement with the first profile to urge the gantry mount along the track to align the latch with the catch in the lock position.

15. The mobile medical imaging system of claim 1, wherein latching system further comprises a biasing member operatively attached to the pedal to bias the pedal toward the engaged position.

16. The mobile medical imaging system of claim 15, wherein the pedal is disposed above a bottom portion of the gantry mount when the pedal is in the engaged position, and the bottom portion of the gantry mount is configured to deflect the pedal from the engaged position toward the disengaged position as the gantry mount translates from the second track end toward the first track end.

17. The mobile medical imaging system of claim 15, wherein the pedal is arranged for user engagement in a first direction and the latching system is configured to retain the pedal in the disengaged position in response to a first user engagement with the pedal in the first direction for disengaging the latch; and
wherein the latching system is configured to release the pedal from the disengaged position in response to a second user engagement with the pedal in the first direction for engaging the latch.

18. (canceled)

19. The mobile medical imaging system of claim 17, wherein the latching system further comprises:
a housing defining a first channel extending between a first top end and a first bottom end, a link landing arranged adjacent to the first bottom end, and a second channel extending between a second bottom end arranged adjacent to the link landing, and a second top end connected to the first channel; and
a link extending between a first end operatively attached to the pedal and a second end, wherein the second end of the link travels along the first channel in response to the first user engagement to abut the link landing to retain the pedal in the disengaged position, and wherein the second end of the link travels along the second channel in response to the second user engagement to release the pedal from the disengaged position.

20. The mobile medical imaging system of claim 1, wherein the at least one imaging component comprises a rotor supporting an x-ray source and a detector and disposed within a housing defined by the imaging gantry for rotation around the imaging bore.

21. The mobile medical imaging system of claim 20, wherein the x-ray source includes a fan-beam x-ray source, and the detector includes an array of detectors; and
wherein the rotor rotates around the imaging bore as the translation mechanism drives the gantry mount along the track in the imaging mode to acquire helical scan x-ray CT images of a patient within the imaging bore.

22. (canceled)

23. The mobile medical imaging system of claim 1, further comprising a pedestal mounted to the base adjacent to the first track end and configured to support a patient support above the base.

24. The mobile medical imaging system of claim 1, further comprising:
a robotic arm extending between a base end operatively attached to the imaging gantry and a mount end arranged for movement relative to the base end; and
an end effector attached to the mount end of the robotic arm and configured to support a tool for engaging a target site, wherein the robotic arm is configured to maintain alignment of the tool relative to the target site.

25. (canceled)

26. (canceled)
